(19) United States
(12) Patent Application Publication
(10) Pub. No.: US 2004/0186504 A1
(43) Pub. Date: Sep. 23, 2004

(54) Oral tissue contourer

(75) Inventors: Carl W. Schulter, Memphis, TN (US); Andrew J. Schulter, Memphis, TN (US)

Correspondence Address: STEPHEN M. PATTON, 7881 GROVE COURT EAST, GERMANTOWN, TN, 38138, US

(73) Assignee: Cagenix, Inc.

(21) Appl. No.: 10/802,206

(22) Filed: Mar. 17, 2004

Related U.S. Application Data
(60) Provisional application No. 60/455,629, filed on Mar. 18, 2003.

(51) Int. Cl. A61M 29/00

(52) U.S. Cl. 606/192

(57) ABSTRACT

A tissue contourer for contouring mucosal tissue in preparation for dental implantation or grafting includes a central portion configured to abut an edentulous portion of a maxilla or mandible and configured to contour mucosal tissue overlying the edentulous portion.

### RELATED APPLICATIONS

This application claims priority from U.S. Provisional Patent Application No. 60/455,629, which was filed on Mar. 18, 2003, and is entitled “Tissue Expander and Method of Using”. This application is related to the co-pending application, “Method of Using a Tissue Contourer”, filed contemporaneously herewith, which is incorporated by reference herein for all that it teaches.

### FIELD OF THE INVENTION

This invention relates to oral surgery. More particularly, it relates to the anatomical contouring of tissue for augmentation of the residual alveolar bone structures with grafting procedures. Even more particularly, it relates to contouring the oral mucosal tissue back to normal anatomical contours by inserting an inflatable bladder submucosally or subperiosteally prior to surgical grafting procedures.

### BACKGROUND OF THE INVENTION

The normal topography of the gingival tissue around teeth has interpapillary and root prominence contours which are determined by the alveolar processes present in normal bone.

When a patient loses a tooth or teeth, he also loses the tissue and bone contours that support the tooth or teeth and that give it a natural appearance. This loss results in a recontouring of the tissue and bone to the contours of an atrophic residual edentulous ridge area which is generally round in shape and lacks natural bone and tissue contours.

As the bone continues to be resorbed over time, it continues to change in a direction that is not a straight vertical or horizontal direction but in a much-defined diagonal direction. The maxillary arch will resorb in an upward and lingual direction and the mandibular arch will resorb in a downward and facial direction. The maxillary arch decreases in vertical height and becomes narrower. The mandibular arch also decreases in vertical height but becomes broader. In order to restore normal contours to the tissue and bone, we must first recontour the tissue to normal contours and then graft the residual bone back to its normal contours. In this way, we can regenerate the natural appearance of the tissue and the bone. It is not just a matter of increasing vertical dimension but a complex dimensional change. For grafting procedures to be surgically successful, they need to be covered with vascularized mucosal tissue to aid in bone growth and to prevent infection. It is also necessary to have a tension free primary closure of the incision. If the incorrect contour of the tissue is used, incomplete coverage of the bone graft will occur or a tension in the tissue will occur resulting in a dehiscence of the tissue and exposure of the graft. When these outcomes are present, the patient will loose some of the bone graft material and will allow the site to become infected.

Devices for creating tubular channels in mucosal tissue have been proposed in the past, but have failed for their intended purposes. For example, U.S. Pat. Nos. 4,798,205 and 4,719,918 suggest devices for enhancing the retention for complete dentures.

In the arrangement of the '918 and '205 patents, a void or channel is created in the mucosal tissues along the crest of a totally edentulous ridge. This void or channel extends completely around the maxillary and mandibular arches. A granular form of hydroxyl appetite material is inserted into a channel along the crest of an eroded maxillary or mandibular ridge to increase the volume of the ridge and hence provide better engagement with and retention of a complete denture.

At the time of the '918 and '205 patents, industry expectations were that a semi-rigid HA material, if properly placed between the bone and tissue would support a complete denture and enhance retention. The material would remain in place and help distribute the load evenly.

In the '205 patent, a midline incision is made in the mandible or the maxilla at the mid-crestal line. Two channels are made that extend from the midline along the crest of the bone in both directions, each terminating at an exist incision at the tuberosity on the maxilla and at the retromolar pad of the mandible.

Once the two channels are made, a thread is inserted into each channel through the mid-crestal incision to the posterior incisions. These threads are connected to two opposing ends of an elongated bladder.

Once they have been inserted through the midline incisions, the ends of the threads are drawn through the posterior incisions until they have been completely removed and the bladder is completely inserted into the channels.

Once in this position, the bladder is inflated. The inflated bladder creates the channel in which the HA is subsequently inserted.

Unfortunately, the channel created by this process doesn't prevent the migration of the granules into the mucosal tissues, which in return permits the loss of retention for the complete denture and the tissue dehiscence by the HA granules. This process was ultimately unsuccessful and has been abandoned by the dental practitioners.

What is needed is a tissue contourer for creating vascularized mucosal tissue with normal tissue contours that can be used for surgical grafting procedures. What is needed is a device that is configured to create this tissue in an edentulous space between existing teeth. What is also needed is a tissue contourer having a volume of between 0.5 milliliters and 31.2 milliliters to create a short recontoured tissue pocket having a similar volume. What is also needed is a tissue contourer having a length of between 6.9 millimeters and 71.2 millimeters and configured to be disposed in an edentulous gap of similar length with contourer ends adjacent to and preferably abutting the terminal teeth. What is also needed is a device for creating a recontoured tissue pocket that is configured to be placed in an edentulous gap of one to eight teeth.

It is an object of this invention to provide a device having these capabilities in one or claimed embodiments thereof.

### SUMMARY OF THE INVENTION

In accordance with a first embodiment of the invention, a tissue contourer or expander is provided having an expandable or an inflatable bladder. This bladder may be inserted under mucosal tissue overlying eroded bone and gradually inflated. By gradually inflating the contourer, the overlying mucosal tissue is encouraged to stretch, expand or otherwise increase its surface area to accommodate the inflated bladder and thereby produce a desired surface contour of the mucosal tissue. This encourages growth and an increase in vascularity of the expanded tissue.

As the mucosal tissue grows, its internal tension is relieved. The bladder can be further and successively inflated or expanded as the tissue expands to provide a void, space or chamber between the bone and the mucosal tissue into which (once the tissue contourer is removed) a bone graft, bone growth enhancer, implant or other prosthetic device that supports the contoured tissue to resemble the bone contours that would have existed were it not for tooth loss and bone erosion.

In accordance with a second embodiment of the invention, a tissue contourer for contouring mucosal tissue in preparation for dental implantation or grafting is provided, the contourer including a central portion having a first side configured to abut an edentulous portion of a maxilla or mandible and a second side configured to contour mucosal tissue overlying the edentulous portion; a first end extending from the central portion and configured to abut a first tooth at one end of the edentulous gap; a second end extending from the central portion and configured to abut another tooth at another end of the edentulous gap; and a means for filling the central portion with fluid.

The first end, the second end and the central portion may have an overall length of no more than 71.2 mm, 52 mm, or 26 mm. The contourer may have an internal working volume of no more than 31.2 ml, 22.1 ml, or 5 ml.

In accordance with a third embodiment of the invention, a tissue contourer bladder for contouring mucosal tissue in preparation for dental implantation or grafting is provided, the bladder including an elongate central portion configured to be disposed between a maxilla or mandible and mucosal tissue overlying the maxilla or mandible; a first end configured to be disposed in a first terminal gap; and a second end configured to be disposed in a second terminal gap.

The first and second terminal gaps may be the same terminal gap. The first and second terminal gaps may be in the same quadrant. The first and second terminal gaps may be in adjacent quadrants.

In accordance with a fourth embodiment of the invention, a tissue contourer for contouring mucosal tissue in preparation for dental implantation or grafting is provided, the contourer including a bladder having an elongated central portion with a texture, wherein the central portion is configured to be disposed between a maxilla or mandible and overlying mucosal tissue; a first end configured to be disposed in a first terminal gap; and a second end configured to be disposed in a second terminal gap.

The texture may be defined by a plurality of protrusions extending outward away from a surface of the central portion. The texture may be further defined by a plurality of concavities extending inwardly from the surface of the central portion. The texture may be defined by a plurality of concavities extending inwardly from a surface of the central portion. The texture may be defined by a plurality of elongated concavities or protrusions. The elongated concavities or protrusions may be formed as elongated ovals. The elongated ovals may be disposed at an angle to its adjacent elongated ovals.

In accordance wit ha fifth embodiment of the invention, a tissue contourer for contouring mucosal tissue in preparation for dental implantation or grafting is provided, the contourer including an elongated central portion having a top side, a bottom side, a facial-facing side, and a lingual-facing side, wherein at least one of the four sides is stiffer than the other three sides; a first end configured to be disposed in a first terminal gap at one end of an edentulous gap; and a second end configured to be disposed in a second terminal gap at another end of the edentulous gap.

The bottom side may be stiffer than the top side. The facial-facing and lingual-facing sides may be stiffer than the top side. The elongated central portion may be a polygon in at least one cross-section. The elongated central portion may be a quadrilateral in at least one cross-section. The elongated central portion may be a regular polygon in at least one cross-section. The two ends may be stiffer than the top side.

In accordance with a sixth embodiment of the invention, a tissue contourer for contouring mucosal tissue in preparation for dental implantation or grafting is provided, the contourer including a cylindrical elongated central portion with at least one longitudinal cross-section that is a conic section; a first end configured to be disposed in a first terminal gap at one end of an edentulous gap; and a second end configured to be disposed in a second terminal gap at another end of the edentulous gap.

The conic section may be a circle. The conic section may not change when the tissue contourer is filled with fluid. The elongated central portion may be made of a self-sealing material.

In accordance with a seventh embodiment of the invention, A tissue contourer for contouring mucosal tissue in an edentulous gap in preparation for dental implantation or grafting, the contourer including a central portion configured to contour mucosal tissue overlying the edentulous gap; a first end configured to be disposed in a first terminal gap at one end of the edentulous gap; a second end configured to be disposed in a second terminal gap at another end of the edentulous gap; and a means for filling the central portion with fluid.

The means for filling the central portion with fluid may include a membrane means for limiting fluid leakage from the contourer. The means for filling the central portion with fluid may include an annulus defining an opening through which fluid is introduced into the central portion. The means for filling the central portion may include a means coupled to and between the annulus and the central portion to direct fluid from the annulus to the central portion.

In accordance with an eighth embodiment of the invention, a tissue contourer for contouring mucosal tissue in an edentulous gap in preparation for dental implantation or grafting is provided, the contourer including a central portion configured to contour mucosal tissue overlying the edentulous gap; a first end coupled to the central portion and configured to be disposed in a first terminal gap at one end of the edentulous gap; and a second end coupled to the central portion configured to be disposed in a second terminal gap at another end of the edentulous gap.

The central portion may have a top surface and the central portion may include a plurality of projections disposed on the top surface and extending upwardly therefrom. The plurality of projections may be spaced between 2 and 10 mm apart. The plurality of projections may be disposed to contour papillae in the mucosal tissue. The plurality of projections extend between 1 and 5 mm upward from the top surface.

In accordance with a ninth embodiment of the invention, a tissue contourer for contouring mucosal tissue in an edentulous gap in preparation for dental implantation or grafting is provided, the contourer including a bladder having a central portion configured to contour mucosal tissue overlying the edentulous gap, a first end configured to be disposed in a first terminal gap at one end of the edentulous gap, and a second end configured to be disposed in a second terminal gap at another end of the edentulous gap, and the bladder defines at least one passageway extending therethrough.

The passageway may extend from a top surface through a bottom surface of the bladder.

The means for filling the second embodiment may be selected from the group consisting of a self-sealing portion of central portion and a port

These and other objects, advantages and aspects of the invention will become apparent from the following description. In the description, reference is made to the accompanying drawings, which form a part hereof, and in which there is shown a preferred embodiment of the invention. Such embodiment does not necessarily represent the full scope of the invention and reference is made therefore, to the claims herein for interpreting the scope of the invention.

### BRIEF DESCRIPTION OF THE DRAWINGS

FIG. 1 shows a first embodiment of a tissue contourer.

FIG. 2 shows another embodiment of the tissue contourer of FIG. 1 having a port connected to a bladder.

FIG. 3 shows a side view of the tissue contourer of FIG. 2.

FIG. 4 is a cross-sectional view of the tissue contourer of FIGS. 2-3 taken at Section Line 3-3 in FIG. 3 and showing how the port provides access to the inside of the bladder.

FIG. 5 shows another embodiment of the tissue contourer of the foregoing FIGURES wherein the port includes an elongated tube that is preferably constructed of the same material as the bladder.

FIG. 6 is a cross-sectional view of the tissue contourer of FIG. 5, taken at Section Line 5-5 looking down the end of the tissue contourer where the elongated tube connects to the bladder.

FIG. 7 is an end view of the tissue contourer depicted in FIGS. 5 and 6 showing the bladder's cylindrical form and closed end.

FIG. 8 is a fragmentary view of another embodiment of the tissue contourer of any of the foregoing FIGURES, showing one end of the bladder modified to include a probe-engaging nosepiece.

FIG. 9 is a cross-sectional view of the device of FIG. 8 taken at Section Line 8-8, and shows two nosepiece hollows, one that forms the end of the bladder and one that receives an insertion probe.

FIG. 10 is a side view of the device of FIGS. 8 and 9 showing the insertion probe fitted into a nosepiece hollow.

FIG. 11 is an end view of the device of FIGS. 8-10 showing the convex side of the nose piece, which faces outward from the bladder.

FIGS. 12 and 13 show details of the port arrangement of FIGS. 5-7.

FIG. 12 is a fragmentary view of the tissue contourer showing the port and the end of the bladder to which it is attached, with the annular structure shown in perspective.

FIG. 13 is a cross-sectional view of the fragmentary structure shown in FIG. 12, with the cutting plane extending generally parallel to the longitudinal axis of the tube and the bladder.

FIGS. 14-16 show an alternative valve arrangement useable with any of the ports of any of the tissue contourers disclosed or described herein.

FIG. 14 is a cross-sectional side view of a port having a self-sealing membrane differing from the arrangement of FIGS. 12-13 in the membrane and annulus arrangement.

FIG. 15 is a top view of the port of FIG. 14.

FIG. 16 is a cross-sectional view of the port of FIGS. 14 and 15 taken at Section Line 14-14 in FIG. 14.

FIG. 17 shows a typical prior art hypodermic needle used to fill the ports of FIGS. 2-7 and 12-16 with fluids such as a sterile saline solution.

FIGS. 18 and 19 show an alternative port arrangement with a mechanical check valve that is configured to be used with any of the tissue contourers disclosed herein.

FIG. 20 illustrates a syringe configured to mechanically engage with the ports of FIGS. 18,19, 21 and 22 to introduce fluid into any tissue contourer bladder to which the port of FIG. 19 is coupled.

FIGS. 21 and 22 are top and side cross-sectional views of alternative port arrangements of the port of FIGS. 18 and 19 in which the planar valve element and spring have been replaced with a spherical valve element.

FIG. 23 is a side view of a rectangular bladder of an alternative tissue contourer.

FIG. 24 is a cross-sectional view of the tissue contourer bladder of FIG. 23 showing its square cross-section. The cross-section is taken at Section Line 23-23 in FIG. 23.

FIG. 25 is a side view of the tissue contourer of FIGS. 23 and 24 showing it in its expanded condition.

FIG. 26 is a cross-sectional view of the tissue contourer of FIGS. 23-25 showing the tissue contourer in its expanded condition. The cross-section is taken at Section Line 25-25 in FIG. 25.

FIG. 27 shows another embodiment of the tissue contourer bladder of FIGS. 23-24 in an alternate expanded form.

FIG. 28 shows a cross-sectional view of the tissue contourer of FIG. 27 taken at Section Line 27-27.

FIG. 29 is a perspective view of another embodiment of a tissue contourer bladder having a circular cross-section.

FIG. 30 is a side view of the tissue contourer bladder of FIG. 29.

FIG. 31 is a cross-sectional view of the tissue contourer of FIGS. 29 and 30 taken at Section Line 30-30 in FIG. 30.

FIG. 32 is a side view of the tissue contourer bladder of FIGS. 29-31 shown as it would appear when expanded.

FIG. 33 is a cross-sectional view of the tissue contourer bladder of FIGS. 29-32 taken at Section Line 32-32 in FIG. 32.

FIGS. 34A, 34B, 35, 36, and 37 show another alternative tissue contourer bladder in the form of a generally circular dome with an expandable top.

FIG. 34A is a side view of the tissue contourer bladder.

FIG. 34B is a cross-sectional view of the tissue contourer bladder taken at Section Line 34-34 in FIG. 34A.

FIG. 35 is a plan view of the tissue contourer bladder of FIGS. 34A and 34B.

FIG. 36 is a side view of the tissue contourer bladder of FIGS. 34A and 34B showing the bladder both in an unexpanded (indicated by the dashed line) and an expanded (indicated by the solid line) condition.

FIG. 37 is a cross-sectional view of the tissue contourer bladder taken at Section Line 36-36 in FIG. 36 and showing the bladder both in an unexpanded (indicated by the dashed line) and an expanded (indicated by the solid line) condition.

FIGS. 38, 39, 40, and 41 show another alternative tissue contourer bladder in the form of a generally circular cylinder having generally hemispherical ends.

FIG. 38 is a top view of the bladder.

FIG. 39 is a cross-sectional view of the bladder taken at Section Line 38-38 in FIG. 38.

FIG. 40 is a top view of the bladder showing it in expanded (the solid line) and unexpanded (the dashed line) condition.

FIG. 41 is a cross-sectional view of the tissue contourer bladder taken at Section Line 40-40 in FIG. 40.

FIGS. 42-45 show another alternative tissue contourer bladder in the form of a generally planar and elongate oval. The bladder of FIGS. 42-45 would appear as an elongated oval in plan view as shown in FIG. 38.

FIG. 42 is a side view of the bladder.

FIG. 43 is a cross-sectional view of the bladder taken at Section Line 42-42 in FIG. 42.

FIG. 44 is a side view of the bladder showing it in expanded (the solid line) and unexpanded (the dashed line) condition.

FIG. 45 is a cross-sectional view of the tissue contourer bladder taken at Section Line 44-44 in FIG. 44.

FIGS. 46-49 are plan views of any of the tissue contourer bladders illustrated or described herein showing alternative plan configurations of those bladders in a succession of plan views.

FIGS. 50 and 51 illustrate another configuration of the tissue contourer bladder of FIGS. 34A, 34B, 35, 36, and 37.

FIG. 50 is a side view of the bladder.

FIG. 51 is a cross-sectional view of the bladder showing thickness differences that permit the upper portion to expand more than the lower portion of the bladder. This view is taken at Section Line 50-50 in FIG. 50.

FIGS. 52 and 53 show another configuration of the tissue contourer bladder of FIGS. 27 and 28 having preferred wall thicknesses.

FIG. 52 is a cross-sectional side view of the bladder.

FIG. 53 illustrates the same view as that of FIG. 52, but shows the bladder in an inflated condition.

FIGS. 54, 55, and 56 illustrate an alternative tissue contourer bladder configuration having walls of differing thickness to control expansion.

FIG. 54 is a perspective view of the bladder illustrating its cylindrical shape with generally flat ends and having a longitudinal axis and a Section Line 54-54 extending coplanar with the longitudinal axis.

FIG. 55 is a cross-sectional view of the bladder of FIG. 54 taken along Section Line 54-54.

FIG. 56 is a cross-sectional view of the tissue contourer of FIGS. 54 and 55 taken along the same Section Line as FIG. 55, but showing the tissue contourer bladder in an expanded condition.

FIGS. 57 and 58 are cross-sectional views of another tissue contourer bladder having the outward appearance of the bladder of FIGS. 38-41, but having varying wall thicknesses to control bladder expansion upon inflation.

FIG. 57 is a longitudinal cross-section of the bladder in an unexpanded condition.

FIG. 58 is a longitudinal cross-section of the bladder in an expanded condition.

FIGS. 59 and 60 are cross-sectional views of another tissue contourer bladder having the outward appearance of the bladder of FIGS. 38-41, but having varying wall thicknesses to control expansion upon inflation.

FIG. 59 is a longitudinal cross-section of the bladder in an unexpanded condition.

FIG. 60 is a longitudinal cross-section of the bladder in an expanded condition.

FIGS. 61A, B, C, D, and E illustrate different surface textures with which any of the tissue contourer bladders disclosed herein can be provided.

FIG. 61A is a fragmentary plan view of a bladder with a pattern of circular protrusions or concavities.

FIG. 61B is a fragmentary plan view of a bladder with a pattern of elongate oval protrusions or concavities disposed both parallel and perpendicular to the length of the tissue contourer bladder.

FIG. 61C is a fragmentary plan view of a bladder with a pattern of elongate oval protrusions or concavities disposed diagonally across the tissue expander bladder.

FIG. 61D is a fragmentary plan view of a bladder with a pattern of elongated rectangular protrusions or concavities disposed longitudinally and laterally.

FIG. 61E is a fragmentary plan view of a bladder with a pattern of elongated rectangular protrusions or concavities disposed diagonally along the bladder.

FIGS. 62A, B, and C are fragmentary cross-sections through the wall of each of the bladders of FIGS. 61A, B, C, D, and E, illustrating alternative arrangements of the protrusions and concavities.

FIG. 62A shows the patterns of FIGS. 61A, B, C, D, and E in cross-section in which all the protrusions or concavities are concavities extending inward generally toward the center of the bladder.

FIG. 62B shows the protrusions or concavities extending outward generally away from the center of the bladder.

FIG. 62C shows the protrusions or concavities extending alternately and generally inward toward and outward away from the center of the bladder.

FIGS. 63-65 illustrate an alternative tissue contourer bladder having a generally vertically-extending wall that defines a passageway therethrough.

FIG. 63 is a plan view of the bladder showing the upper end of the passageway.

FIG. 64 is a cross-sectional side view of the bladder taken at Section Line 63-63 in FIG. 63 showing the vertically extending walls of the passageway.

FIG. 65 is a side view of the bladder.

FIGS. 66-67 illustrate an alternative tissue contourer bladder having a plurality of contouring projections extending upward from its upper surface.

FIG. 68 is a plan view of an alternative tissue contourer bladder constructed according to that of FIGS. 66-67, having four generally vertical passageways extending therethrough that are defined between each of four upwardly extending contouring projections.

FIG. 69 is a cross-sectional view of the tissue contourer of FIG. 68 taken at Section Line 68-68 in FIG. 68.

FIGS. 70 and 71 show a connector for joining two bladders to a single port for joint and simultaneous filling and emptying.

In many of the embodiments illustrated in the FIGURES above, the walls of the tissue contourer bladder are shown in cross-section and are shown as having a significant cross-sectional thickness with respect to the overall dimensions of the tissue contourer bladder itself. The actual thickness of the walls can vary widely as recited herein. It is only by showing walls with a significant thickness in the FIGURES that the applicants are able to show the cross hatching at all

### DETAILED DESCRIPTION OF THE PREFERRED EMBODIMENTS

While the present invention is susceptible to being embodied in various forms, there is shown in the drawings a presently preferred embodiment hereinafter described. It should be understood that the present disclosure is to be considered as but one example of the invention, provided in sufficient detail for those skilled in the art of dental devices to construct. It is not intended to limit the invention to the specific embodiments illustrated.

In the discussion below, several terms are used in a particular manner as explained in the following paragraphs.

An “edentulous gap” is defined herein as the space between a first tooth (or tooth substitute) and a second tooth (or tooth substitute) when all the in-between teeth are removed; or the space between a first tooth (or tooth substitute) and a tuberosity (or retromolar pad) when all the in-between teeth are removed.

A “terminal gap” is defined herein as that portion of an edentulous gap that is adjacent to an existing tooth, tooth substitute, retromolar pad, or tuberosity and within a distance of a single missing tooth of the existing tooth, tooth substitute, retromolar pad, or tuberosity.

An “edentulous void” is defined as a space formed between a ridge and the overlying mucosal tissue in a space previously occupied by a tooth.

A “terminal void” is defined herein as that portion of an edentulous void that is adjacent to an existing tooth, tooth substitute, retromolar pad, or tuberosity and within a distance of a single missing tooth of the existing tooth, tooth substitute, retromolar pad, or tuberosity.

“Terminal tooth” is defined as the still-existing tooth (or tooth substitute) that defines an end of an edentulous gap.

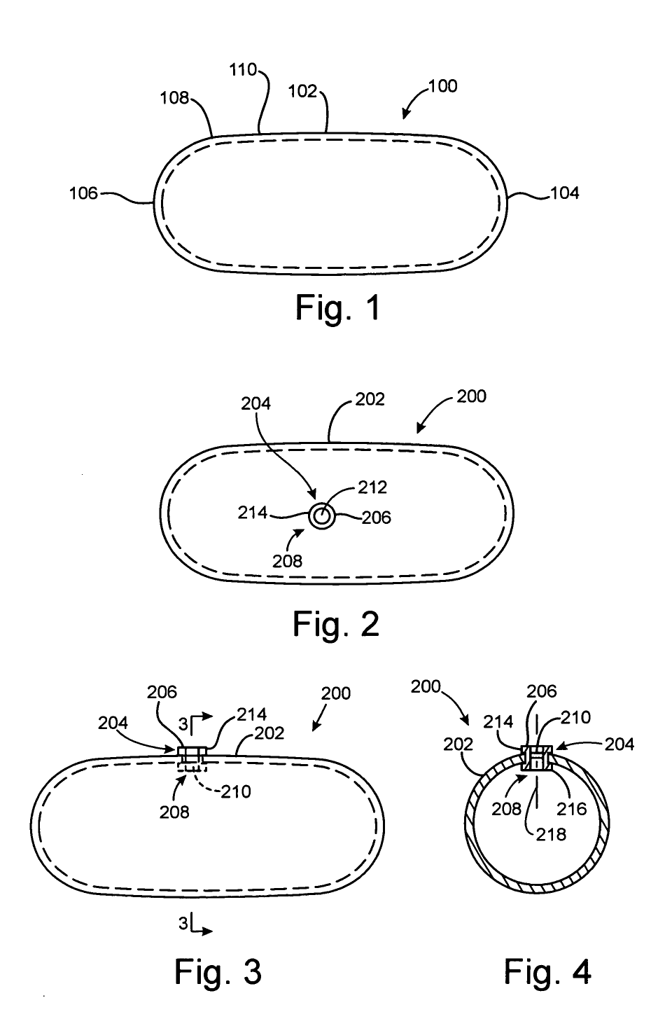

Referring now to the drawings, wherein like reference numerals refer to like parts throughout the several views, there is shown in FIG. 1 a tissue contourer 100 in the general form of a hollow cylinder 102 enclosed by generally hemispherical ends 104 and 106. The cylindrical portion and the ends define an enclosed bladder 108 defined by a wall 110 of a generally constant thickness. The preferred wall thickness is between 0.01 and 1.0 mm. The length of the bladder 108 is preferably between 2 and 250 mm, which will accommodate a range of edentulous gaps between adjacent teeth. The outer cylindrical diameter of the bladder is preferably between 2 and 20 mm.

The material from which the bladder is formed is self-sealing after it is pierced and the bladder filled. To fill the bladder, the operator inserts a thin tube such as a hypodermic needle through the wall of the bladder and introduces fluid into the bladder. This causes the bladder to expand. When the operator withdraws the thin tube from the bladder, the hole through which the tube passed self-closes and prevents significant fluid leakage from the bladder.

Preferably, at least 10% of the bladder surface is configured to self-seal when pierced and filled as described above. With an at least 10% surface area available for piercing and filling the bladder, the operator can pierce the mucosal tissue from any of several locations. These multiple sites permit the operator to inject fluid through the mucosal tissue into the bladder from several different locations, reducing the change for inflammation and infection resulting from the overuse of any particular location. In a more preferred embodiment, at least 25% of the bladder is self-sealing. In another preferred embodiment, the self-sealing portion of the bladder is located on an upper portion thereof.

FIGS. 2, 3 and 4 show another embodiment 200 of the tissue contourer 100 of FIG. 1 that is constructed and configured in exactly the same manner as contourer 100, but includes a specially configured region through which the operator insert fluid into the bladder. This region is called an “access port” or (more generally) a “port” herein.

In the embodiment of FIGS. 2, 3, and 4, tissue contourer 200 has a bladder 202 with an access port 204 on top of the bladder. The “top” of the bladder refers that portion of the surface of the bladder that faces toward the mucosal tissue and away from the maxilla or mandible when the tissue contourer is placed between the mucosal tissue and the bone of the mandible or maxilla.

Port 204 is provided to permit the operator to introduce fluid into (and preferably remove fluid from) the bladder. In the embodiment shown in FIGS. 2, 3, and 4, port 204 includes a circular annulus 206 that is disposed in circular opening 208 located on top of bladder 202.

Port 204 also includes a membrane 210 that extends across a central opening 212 of annulus 206 and closes it off. Membrane 210 is self-sealing. The operator can insert a hollow tube, such as a hypodermic needle, through the membrane and fill bladder 202.

Membrane 210 prevents fluid from leaking out of the bladder through annulus 206. By providing bladder 202 with a port having self-sealing or self-closing capabilities such as that provided by membrane 210, fluid insertions into the bladder through the membrane 210 can be retained inside and prevented from leaking back out through the port. This permits a manufacturer to make bladder 202 out of different materials with different characteristics than might be required if the bladder material itself was required to be self-sealing. The wall thickness can thereby be made thinner, thicker or of different materials than the membrane 210.

Annulus 206 has an outer flange 214 and an inner flange 216. The outer flange 214 extends radially outward away from the longitudinal axis 218 of the annulus 206 across the outside surface of the bladder 202. Flange 214 is generally parallel with the outer surface of the bladder in the region of the annulus 206.

Inner flange 216 extends radially outward away from the longitudinal axis 218 of the annulus on the inside of the bladder and generally parallel with the inner surface of the bladder 202 in the region of the annulus 206.

Port 204 is mounted generally flush with the surface of the bladder 202 and in a central longitudinal region thereof. It extends no more than 3 mm above the top surface of the bladder, more preferably no more than 2 mm, and even more preferably no more than 1.25 mm. It should be recognized that the bladder 202 of FIGS. 2, 3, and 4 may be made in the same manner and with the same construction and characteristics as bladder 108 of FIG. 1, and that bladder 108 may be made in the same manner and with the same construction and characteristics as bladder 202.

Port 204 need not be located on the surface of the bladder, nor need it be located generally in a central longitudinal region of bladder 202.

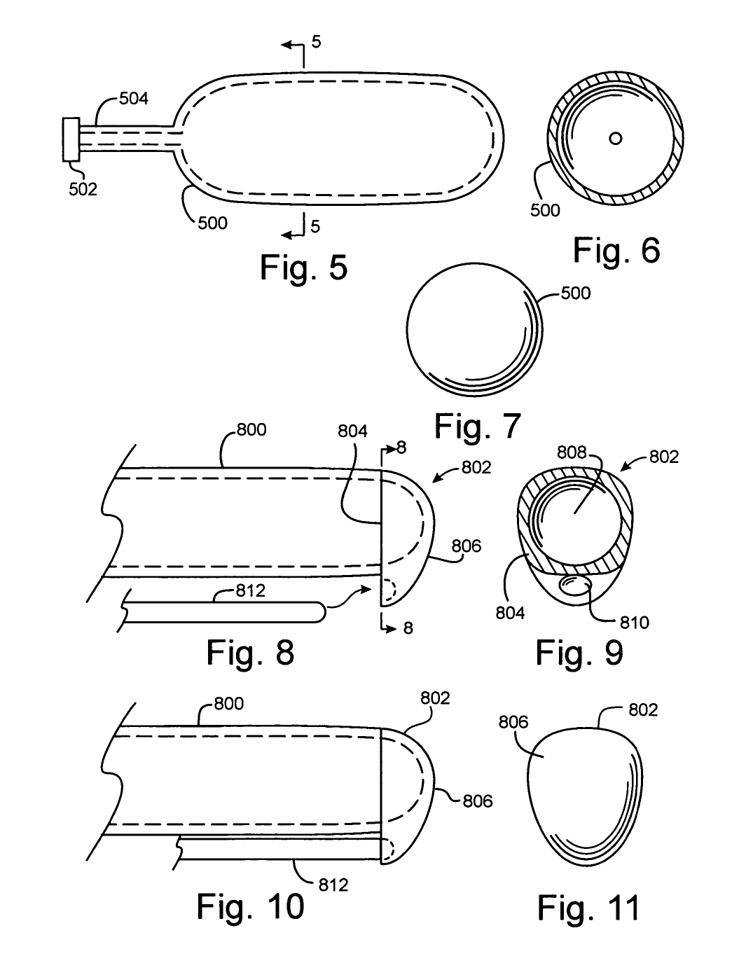

FIGS. 5, 6 and 7 show an alternative embodiment of the tissue contourer of the foregoing FIGURES in which the port 204 has been moved to a different location that is adjacent the end of the bladder of the foregoing FIGURES, which is identified in FIGS. 5, 6, and 7 as bladder 500. The only difference between the device of FIGS. 5, 6, and 7 and the devices of the foregoing FIGURES is in the location of the port, identified in FIGS. 5, 6, and 7 as port 502.

Port 502 of the tissue contourer embodiment of FIGS. 5, 6, and 7, has the same construction as port 204 of FIGS. 2, 3, and 4. It is located differently, however. Port 502 also includes a tube 504 that extends from one end of bladder 500 at the point where the longitudinal axis of bladder 500 intersects the bladder itself. Tube 504 preferably has a length of between 1 and 25 mm.

Tube 504 permits access to the bladder 500 without the need to insert a needle or other filling device through the mucosal tissue and into the port, as would be the case using the embodiment of FIGS. 2, 3, and 4. In this way, a tube extending through an opening or aperture in the mucosal tissue can fill the bladder 500.

Tube 504 is in fluid communication with both the annulus and the bladder to conduct fluid from the annulus to the bladder, and preferably to conduct it in the opposing direction as well. The length of the tube is preferably between 1 and 10 mm, and is preferably of the same material as that used to form the bladder 500.

FIGS. 8, 9, 10 and 11 illustrate an alternative bladder end configuration for any tissue contourer bladder described or illustrated herein. This alternative bladder end is usable with any of the foregoing tissue contourers by configuring an end of the bladder with nosepiece 802.

The nosepiece can either replace a bladder end and thereby become a part of the bladder itself, or it can be fixed to bladder end as an attachment thereto. It is preferably made of the same material as the bladder itself.

Referring to FIG. 8, a bladder 800, which is representative of any of the bladders described herein, includes a nosepiece 802 in the general form of a prolate hemispheroid that is flattened on one end. It has two sides, a first concave side 804 that faces the bladder and the probe, and a second convex side 806 that faces outward in the form of a prolate hemispheroid that is slightly flattened along its major axis.

The nosepiece has two hollows on the concave side. Hollow 808 is configured to receive (or to form) the end of the bladder, and hollow 810 is configured to receive the end of the probe.

To use the contourer, the operator inserts an insertion probe 812 into hollow 810 of nosepiece 802. The operator then pushes the end of the probe together with tissue contourer into an incision that passes through the mucosal tissue. As the probe is forced into the incision and along the maxilla (or mandible, if the bladder is being used to contour mandibular mucosal tissue), the end of probe 812, which is engaged in hollow 810, pulls the tissue contourer along with it until the tissue contourer is drawn through the incision and between the bone and the overlying mucosal tissue. Once in this position, the probe can be withdrawn from the incision leaving the tissue contourer behind. The operator can then fill the tissue contourer with fluid and begin contouring the skin.

Note that probe 812 is disposed generally parallel and adjacent to the longitudinal extent of the tissue contourer bladder of FIGS. 8 and 10. It is in this parallel and adjacent orientation that the probe inserts the tissue contourer underneath the mucosal tissue.

In FIGS. 8-11 the bladder is shown as having a hollow 810 that engages the probe tip. In alternative structures, the relations could be reversed, such as by having a hollow probe engage a protrusion located where hollow 810 is shown. In FIGS. 8-11, the probe engages the bladder end directly. In an alternative embodiment, a latch, catch, pin, clasp, link, clip, detent, or other releasable structure for engaging the probe and the bladder end may be provided to couple the two together.

Port Arrangements

FIGS. 12 and 13 show an alternative port 1200 that may be used in any of the tissue contourers discussed herein. It includes an annular structure 1202 in fluid communication with a tissue contourer bladder 1204 (which may be any of the bladders illustrated or described herein). Annular structure 1202 includes a semi-rigid annulus 1206 defining a central longitudinal passageway 1208 across which is fixed a membrane 1210 that is self-sealing. Membrane 1210 extends across the central longitudinal opening of the annulus 1206 and seals the opening, thereby preventing fluid from escaping from the bladder.

A tube 1212 is coupled to annulus 1206 to conduct fluid from the annulus to the bladder. Annulus 1206 is fixed to the end 1209 of tube 1212 to limit and preferably block the flow of fluid therethrough, but to permit an operator to insert a hypodermic needle or other tubular structure therethrough to fill the bladder with fluid.

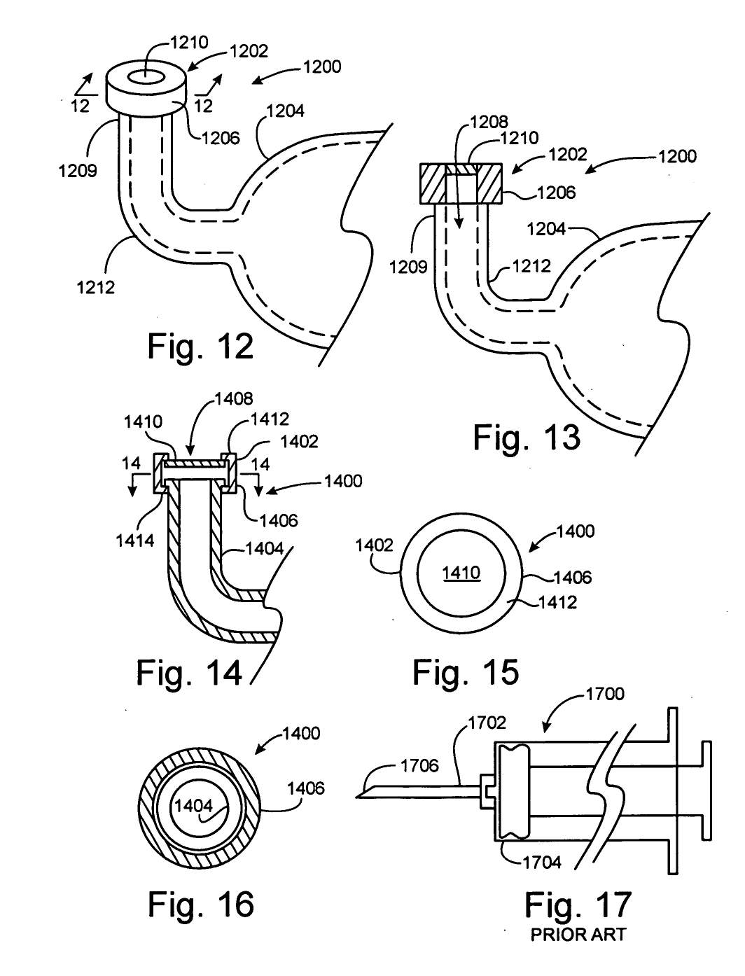

FIGS. 14, 15 and 16 show an alternative port 1400 that differs from the arrangement of FIGS. 12-13 in the structure of the annulus and the arrangement of the membrane.

Port 1400 includes an annular structure 1402 in fluid communication with a tube 1404 that is in fluid communication with a tissue contourer bladder (not shown). Port 1400 functions to reduce or prevent the flow of fluid out of the bladder. The annular structure 1402 is coupled to and in fluid communication with both tube 1404 and bladder (not shown) to conduct the fluid that fills the bladder therebetween.

The annular structure 1402 includes a semi-rigid annulus 1406 defining a central longitudinal passageway 1408 across which is fixed a membrane 1410 that is self-sealing. Membrane 1410 is circular and extends perpendicular to and across passageway 1406 to seal the passageway, thereby limiting or preventing fluid from escaping from the bladder.

The annulus includes upper and lower inwardly extending flanges 1412 and 1414, respectively, to which the membrane and the tube are attached. The membrane 1410 is bonded to the inside surface of flanges 1412 and the tube is bonded to the inside surface of flange 1414.

FIG. 17 illustrates a prior art syringe 1700 used to fill any of the ports having self-sealing membranes described herein. The syringe includes a needle 1702 that is mounted to the syringe body 1704. Needle 1702 has a point 1706 that is configured to pierce and penetrate the self-sealing membranes of the ports shown herein. It is also configured to pierce any self-sealing bladder of a tissue contourer described herein.

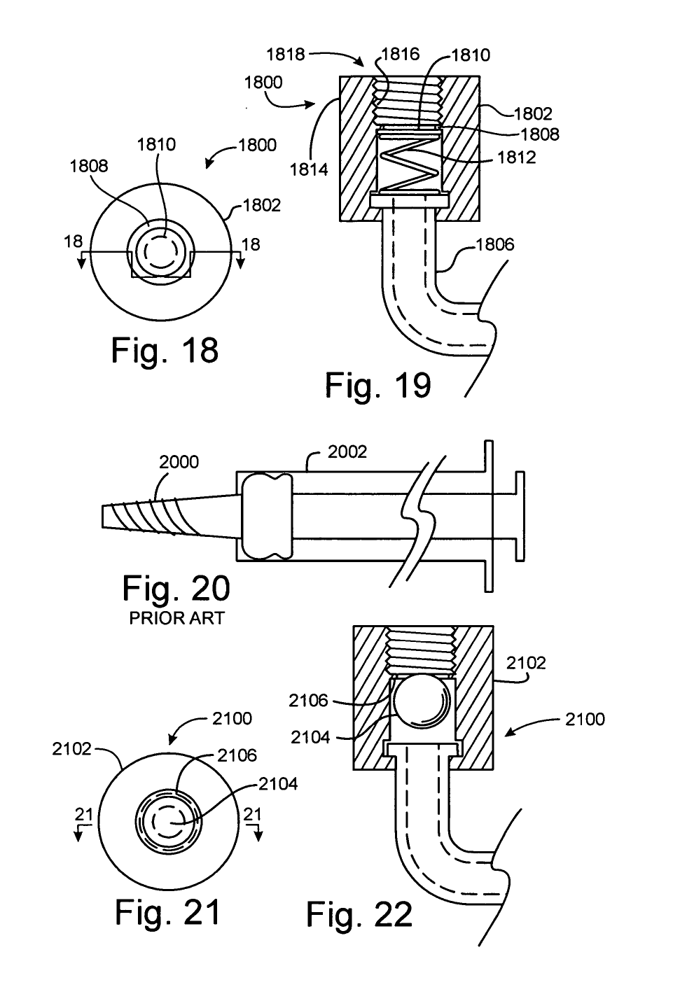

FIGS. 18 and 19 show another alternative port 1800 that may be used in any of the tissue contourers discussed herein. It includes an annular structure 1802 in fluid communication with a bladder (not shown). A tube 1806 is coupled to and in fluid communication with both annular structure 1802 and bladder to conduct fluid to and from the bladder.

Annular structure 1802 has an inwardly extending circular valve seat 1808. Structure 1802 includes a circular planar valve element 1810 having a diameter that is greater than that of seat 1808. Valve element 1810 is configured to abut seat 1808 and to block off fluid flow through tube 1806, thereby preventing fluid from leaking out of the bladder to which the tube is connected.

A spring 1812 is disposed within annular structure 1802 with one end abutting the annular structure and the other end abutting valve element 1810. The spring is configured to press valve element 1810 against seat 1808. The spring is preferably a metallic coil compression spring, slightly compressed when valve element 1810 is closed in order to keep the valve element sealed against seat 1808. The spring may also be made of plastic. Rather than being separately manufactured and inserted into the annular structure, the spring can be formed integrally with the annular structure. It may also be configured as a leaf spring or a tension spring.

Tube 1806 is coupled to and in fluid communication with a tissue contourer bladder, which may be any of the tissue contourer bladders described herein.

Annular structure 1802 includes an upper portion 1814 that is configured to mechanically engage a syringe (FIG. 20) or other structure for inserting fluid into the port to the tissue contourer to which it is coupled. In the preferred embodiment, the upper portion 1814 of the valve body has a threaded internal surface 1816 that engages a similar threaded outer surface 2000 of syringe 2002 (FIG. 20). To couple syringe 2002 and annular structure 1802, the operator inserts the threaded outer surface 2000 into a cylindrical hole 1818 in upper portion 1814. The operator then twists syringe 2002 with respect to the upper portion 1814 until the threads on the syringe 2002 and the annular structure 1802 mutually interengage to create a leak-resistant joint between the syringe and the port 1800.

The threaded portions of the valve body and the syringe are preferably provided with a thread pitch and taper sufficient to engage and seal one against the other after five revolutions of the syringe with respect to the valve body or less, more preferably after three revolutions or less, and even more preferably after two revolutions or less.

FIGS. 21 and 22 illustrate the port of FIGS. 18 and 19 having a modified annular structure 2102. The modified annular structure 2102 of port 2100 is configured to accommodate a movable spherical ball valve element 2104. Valve element 2104 abuts and seals against valve seat 2106 formed on an interior surface of annular structure 2102. Other than the replacement of the valve element 1810 and spring 1812 with the ball valve element 2104, port 2100 shown in FIGS. 21 and 22 is the same in all respects as port 1800 shown in FIGS. 18 and 19.

Bladder Arrangements

The bladders of the tissue contourers shown in the above examples are generally configured as elongated circular cylinders having generally hemispherical ends. Bladders configured in this manner are suitable for general use. Other configurations of these bladders are possible and indeed recommended for particular applications to expand tissue.

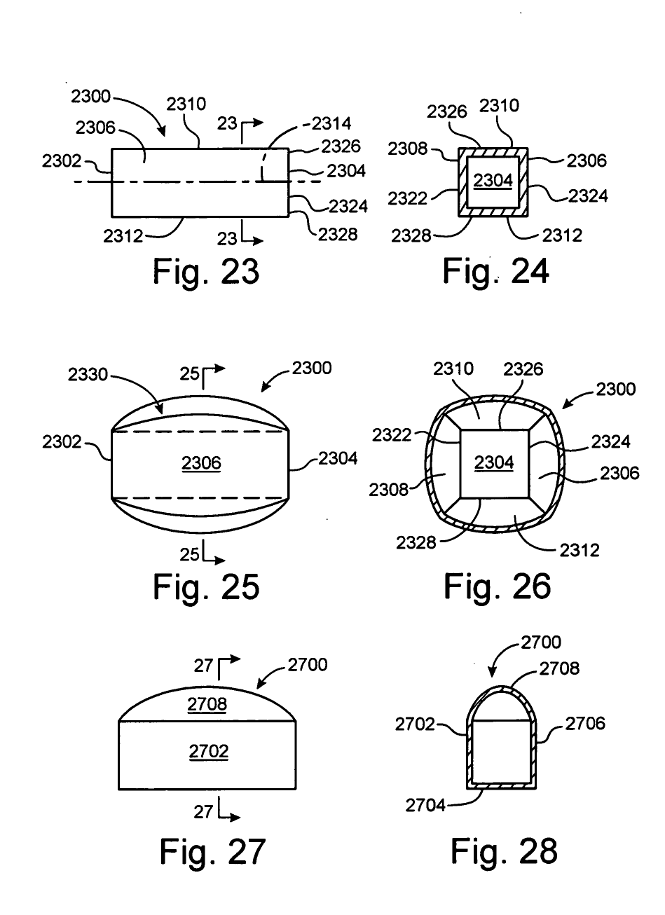

FIGS. 23-60 illustrate several of these alternative bladders that are constructed somewhat differently from the bladders identified above. These bladders differ from the bladders illustrated above in the stiffness, thickness and relative elasticity of their walls, as well as their overall geometry and shape, discussed in more detail below. In each of these embodiments, at least one wall and often more than one are stiffer than other walls as indicated by their ability to resist bending or expanding more than other walls. This stiffness may be provided by making walls out of different materials, having different thicknesses or both.

As in the case of the bladders shown and discussed above, they also can be made of the same materials and may be equipped with a port such as any of the port arrangements described above.

FIGS. 23-26 show the first of these alternative bladder arrangements. In these FIGURES, the bladder 2300 is an elongated body, having two opposing ends 2302, 2304, each end having a smaller area than the elongated sides 2306, 2308, 2310, and 2312.

The bladder is shown (FIG. 24) in a cross-section that is taken perpendicular to the longitudinal axis 2314 of bladder 2300. In this cross-section, the bladder 2300 is a polygon having four sidewalls or sides 2306, 2308, 2310, and 2312. Two of those cross-sectional sides, the top side 2310 and the bottom side 2312, are parallel. Two other sides, the left side 2306 and the right side 2308, are also parallel. The top and bottom sides 2310, 2312 are of equal length. The left and right sides 2306, 2308 are of equal length. Each of the four side walls 2306, 2308, 2310, 2312 are disposed at right angles to their adjacent sidewalls.

Ends 2302, 2304 of the bladder are polygons and have four edges where ends 2302, 2304 meet their adjacent sidewalls 2306, 2308, 2310, and 2312. The edges where end 2304 meets its sidewalls 2306, 2308, 2310, and 2312 are identified as items 2322, 2324, 2326, and 2328.

The top and the bottom edges 2326 and 2328 of end 2304 are parallel. The left and the right edges 2322 and 2324 of end 2304 are also parallel. The ends 2302, 2304 are parallel to each other, and are preferably polygons, preferably regular polygons, preferably rectangles, and preferably squares.

FIGS. 25 and 26 illustrate the manner in which bladder 2300 is inflated to increase its size. In FIGS. 25 and 26, the bladder expands in a central region by stretching the four sides 2306, 2308, 2310, and 2312. When the bladder is inflated, these sides stretch and expand outward.

As the bladder is inflated, the walls are configured to expand such that middle region 2330 of bladder 2300 becomes generally circular in longitudinal cross-section and the edges between each adjacent side 2306, 2308, 2310, and 2312 flatten.

The walls are also configured to expand during inflation such that the middle region 2330 has the greatest girth and the greatest increase in size. The girth of the bladder preferably decreases as one moves from the central region 2330 toward each end 2302, 2304.

The walls of bladder 2300 are also configured such that successive longitudinal cross-sections of inflated bladder 2300 (as shown in FIGS. 25 and 26) are successively more square and the edges between adjacent sides 2306, 2308, 2310 and 2312 more pronounced as one moves along bladder 2300 from central region 2330 toward either opposing end 2302, 2304.

In FIGS. 25 and 26, the ends 2302, and 2304 are configured to flex less than the sides, therefore causing them to remain generally flat as compared to sides 2306, 2308, 2310, and 2312 when the bladder is inflated. As bladder 2300 is inflated, the ends neither substantially stretch, nor increase in size (as compared to adjacent sides 2306, 2308, 2310 and 2312) and maintain substantially their original dimensions (as compared to adjacent sides 2306, 2308, 2310 and 2312) even after the bladder is inflated.

One way of making the ends stiffer such that they deflect less than sides 2306, 2308, 2310, and 2312 when the bladder is inflated is to make the ends 2302, 2304 thicker than the sides. Increased stiffness may also be provided by making ends 2302, 2304 of a material different than that of sides 2306, 2308, 2310, 2312, a material having a greater modulus of elasticity than that of the sides. The material of which the ends are constructed may be filled or reinforced with particles, threads or fibers of a material having a higher modulus of elasticity than that of sides 2306, 2308, 2310, and 2312.

In some cases, it may be desirable to employ a tissue contourer that is configured to enlarge or expand on one side more than another. For example, a bladder providing greater expansion of its top side than its left side, right side and bottom, or of its top, left and right sides than its bottom side. With bladders configured in this manner, the bladder's bottom side (which is installed adjacent to bone of the mandible or maxilla) would remain in a more accurate position, and be less inclined to slip when the patient chews, for example.

A first example of such a heterogeneously expanding tissue contourer bladder can be seen in FIGS. 27 (side view) and 28 (cross-section), which shows the tissue contourer in is expanded condition. In its unexpanded condition, it would appear identical to the bladder of FIGS. 23 and 24.

Bladder 2700 of FIGS. 27 and 28 differs from the bladder 2300 of FIGS. 23, 24, 25 and 26 in one respect only: three of its longitudinally extending left, bottom and right sides 2702, 2704, 2706 are constrained to expand and flex outward less than the longitudinally extending top side 2708 when the bladder is inflated.

The greater relative stiffness of sides 2702, 2704, 2706 is provided by making sides 2702, 2704, 2706 thicker than side 2708. It may also be provided by making sides 2702, 2704, and 2706 of a material different than that of side 2708, a material that, for example, has a greater modulus of elasticity than the modulus of elasticity of sides 2702, 2704, and 2706. The material used on sides 2702, 2704, 2706 may also be reinforced, such as by embedding fibers, threads or particles in the material of which they are comprised.

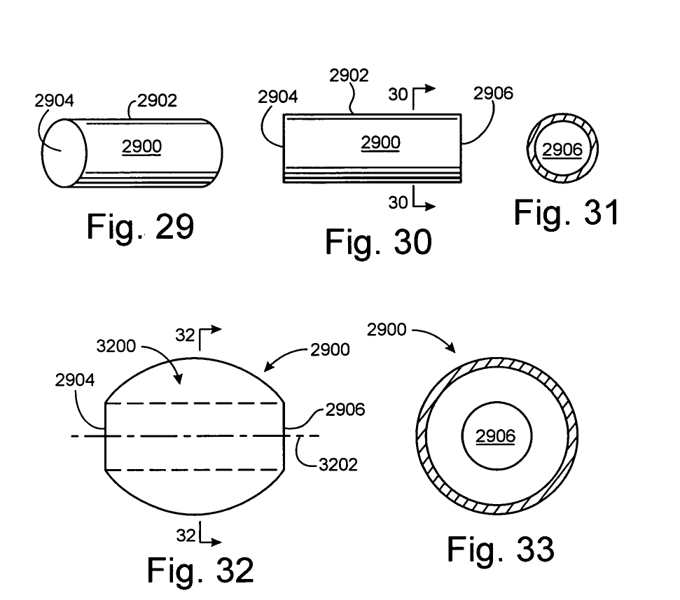

FIGS. 29-33 show another embodiment of the tissue contourer identified as item 2900. Bladder 2900 is generally in the form of an elongate cylinder 2902 having relatively flat ends 2904 and 2906. In cross-section, cylinder 2902 is not a polygon. It is a closed conic section, as shown in the cross-section of FIG. 31. Preferably the conic section is a circle. FIG. 31 also illustrates another characteristic of bladder 2900, that the wall thickness of the bladder is constant as one moves in a circumferential direction.

Cylinder 2902 is a right circular cylinder and has a constant cross-sectional area. Ends 2904 and 2906 of bladder 2900 therefore have the same area and are both conic sections, preferably circles. Ends 2904 and 2906 are also both preferably parallel, to each other.

FIGS. 32 and 33 illustrate the bladder 2900 of FIGS. 29, 30 and 31 after inflation. The bladder 2900, when filled with fluid, expands along its length to form the cylindrical shape shown in FIGS. 32 and 33. In this configuration, the cylinder has a greater cross-sectional area at the center portion 3200 of its longitudinal axis 3202 than it does at each end 2904, 2906. The cross-sectional profile is circular.

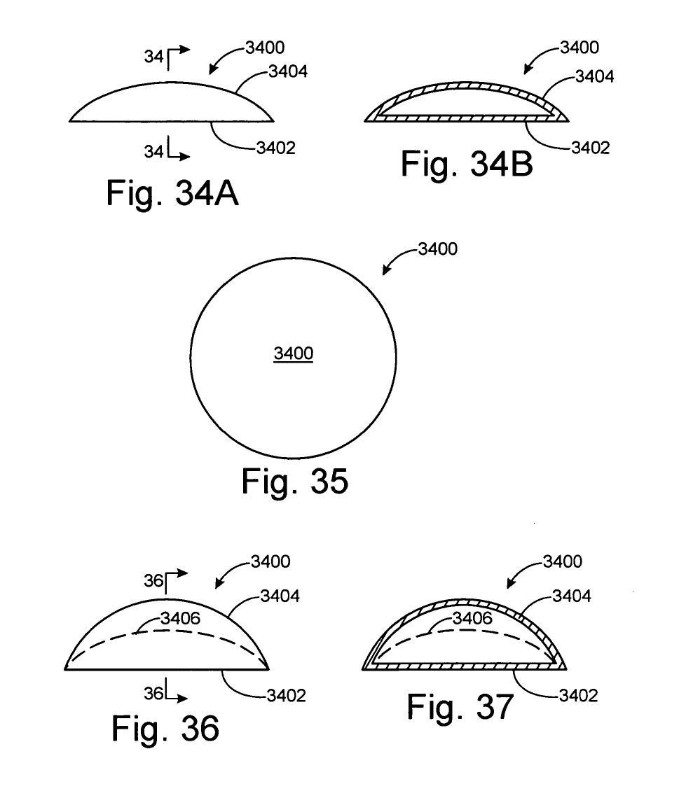

FIGS. 34A, 34B, 35, 36, and 37 illustrate an alternative tissue contourer bladder 3400 having a planar lower wall 3402 and a convex upper wall 3404 bonded to the lower wall 3402 at the distal edges of the upper and lower walls to form a generally lens-shaped bladder.

The lower wall 3402 is more rigid than the upper wall 3404. When bladder 3400 is inflated, upper wall 3404 expands and moves upward, increasing in size. At the same time, the lower wall 3402 maintains its planar area and planar shape constant.

In plan view, the shapes of both the upper and lower walls are circular as shown in FIG. 35. This shape does not change when the bladder is inflated, as best seen in FIG. 36 and (in cross-section) FIG. 37.

In FIGS. 36 and 37, the curved dashed line 3406 represents the original, unexpanded position of the upper surface. The curved solid line represents the position of the upper wall after the bladder has been filled and the upper wall has been distended and expanded.

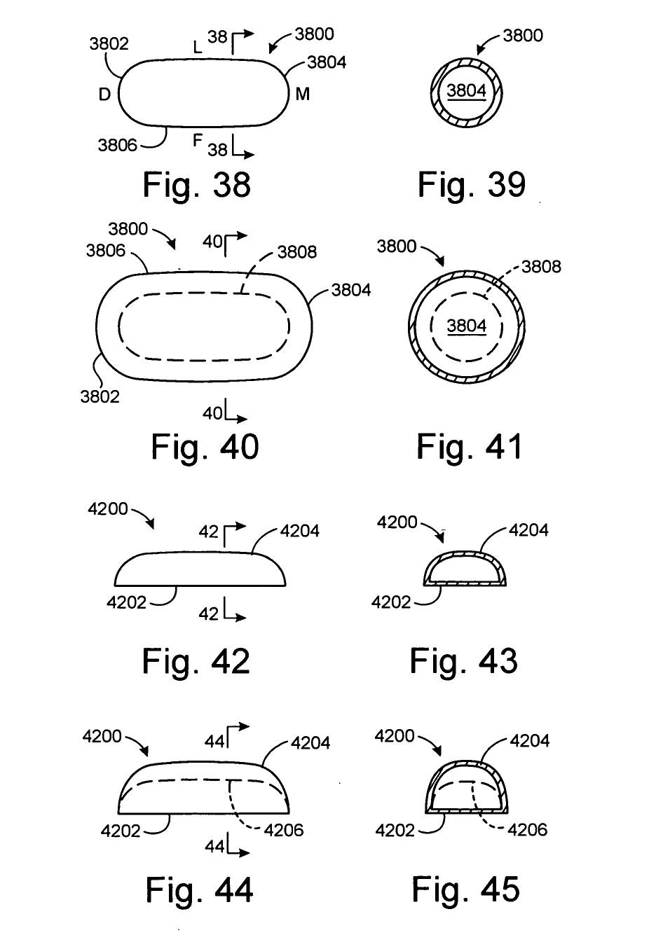

FIGS. 38-41 illustrate another configuration of the tissue contourer bladder in which the bladder 3800 has two hemispherical ends 3802 and 3804 that are coupled to an elongated circular cylindrical portion 3806. In this example, when bladder 3800 is filled, the cylindrical portion 3806 is filled; it expands generally uniformly, maintaining a relatively constant diameter over substantially all of its length. In a similar fashion, the hemispherical ends also expand uniformly maintaining their hemispherical shape as the size of the tissue contourer bladder increases.

This increase in size is apparent from an examination of FIGS. 40 and 41 (which is a cross-section of FIG. 40). Dashed line 3808 in FIGS. 40 and 41 represents the outer surface of the bladder before it is filled and expanded. The solid line in FIG. 40 represents the outside surface of that same bladder 3800 after it has been filled and expanded. Note that the diameter of cylindrical portion 3806 is preferably constant along its length.

The overall length of the bladder has increased as well. This increase is length is provided by the expansion and outward distension of ends 3802 and 3804.

FIGS. 42-45 show another embodiment 4200 of a tissue contourer bladder having an elongated shape with generally rounded ends, a flat bottom and a rounded upper surface.

The bladder 4200, shown in cross-section in FIG. 43, has a bottom wall 4202 of relatively constant thickness and a top wall 4204 of relatively constant thickness as well. The two walls meet at their distal edges where they are joined together to create the enclosed bladder shape.

The top wall 4204 is convex, and arches upward across the top of the bottom wall 4202. The length of bladder 4200 is greater than its width.

FIGS. 44 and 45 illustrate the same side view and the same cross-sectional view of bladder 4200 that FIGS. 42 and 43 illustrate. FIGS. 44 and 45 illustrate bladder 4200 in its expanded or distended condition after it has been filled with fluid.

In FIGS. 44 and 45, the original shape of the top wall of the bladder is shown as dashed line 4206. The new shape and position of the top wall of the bladder is shown as a solid line.

A comparison of the two illustrates that the bottom wall of the bladder is rigid and planar even after the bladder has been filled with fluid. It is stiff enough to prevent the bladder from increasing in length or in width as bladder 4200 is filled with fluid and expands.

FIGS. 46, 47, 48, and 49 illustrate alternative tissue contourer bladder configurations 4600, 4700, 4800, and 4900 that differ from the bladders of FIGS. 34-45 in one respect only: their shape in top or plan view. Like the embodiments of FIGS. 34-45, bladders 4600, 4700, 4800, and 4900 have flat bottom walls and curved top walls 4602, 4702, 4802, and 4902 that expand or distend upward when the bladders are filled with fluid. They also have the same planar bottom wall construction that is stiff and does not distend or expand when the bladder is filled with fluid as the top wall expands.

The bladder of each of FIGS. 46, 47, 48 and 49 is intended for insertion in the mouth of a patient in a particular orientation given by the letters F (facial), L (lingual), D (distal) and M (mesial). Thus, the bladders of FIGS. 46, 47 and 48 have a greater lingual-facial width at their distal end than they have at their mesial end.

This difference in width provides more tissue contouring for the distal teeth (e.g. cuspids) than it does for the mesial teeth (e.g. incisors) where the crest curvature is greatest.

Another feature of the bladders of FIGS. 46 and 47 is the curvature of the bladder in the distal-mesial direction. The bladder curves to follow the crest. This insures that the bladder will contour the tissue at the appropriate location.

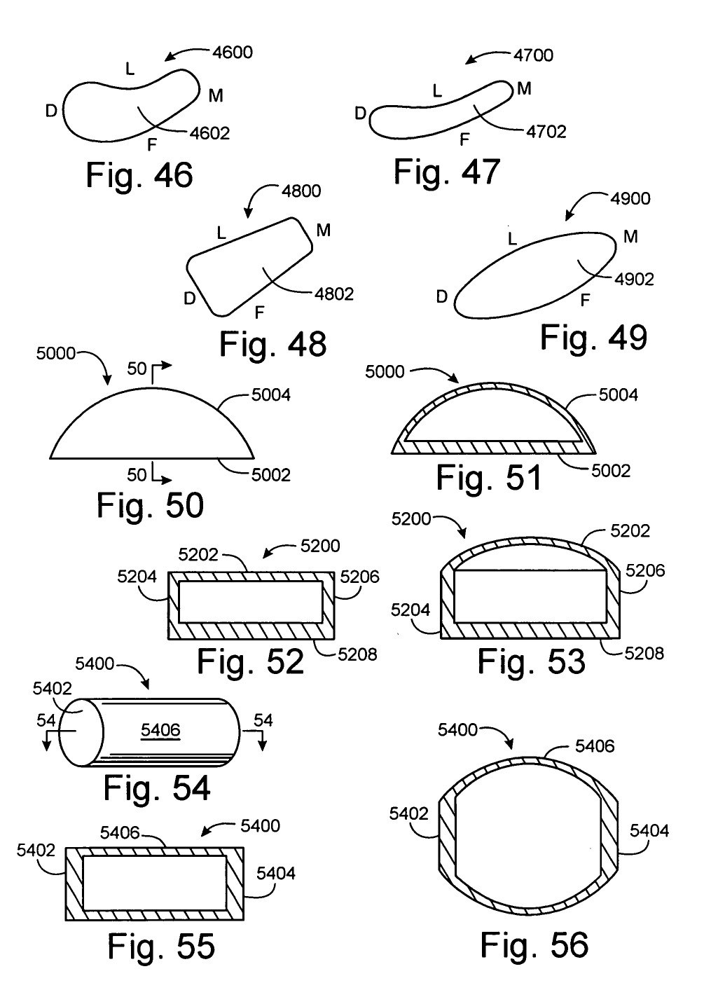

Bladder 4800 of FIG. 48 has a broader distal end and narrower mesial end, but lacks the curvature shown in the examples of FIGS. 46 and 47. This bladder, in fact, is generally straight and does not curve along its distal/mesial axis. It is intended for molar/premolar contouring.

Bladder 4900 of FIG. 49 is configured to be broader in the middle of its length in the facial direction and than it is at either end. This configuration would be particularly beneficial when dealing with severe erosion. In cases such as these, the erosion is most severe in the central region of the edentulous span and the tissue contourer must have a greater thickness in the ventral region raise the receded gum tissue farther to its original gum line.

Wall thickness

Varying the thickness of the bladders can provide increased stiffness and reduced expandability in different regions of the bladder itself. This is important where one desires more tissue expansion in one region of the mucosal tissue and less in another.

When a tissue contourer with a varying wall thickness is filled with fluid, the thicker regions do not expand as much as the thinner regions. The regions that expand more place additional pressure on the mucosal tissue, which causes it to expand more.

More tissue expansion is provided in the portions of mucosal tissue that surround the thinner-walled regions of the tissue contourer bladder than those that surround the thicker-walls regions of the tissue contourer bladder.

Thus, if one wants to encourage greater tissue expansion in the region of a particular missing tooth, for example, one would employ a tissue contourer having thinner walls adjacent to the region of the missing tooth.

Due to the thinner walls in this region, the contourer would expand to a greater degree and additional expanded tissue would be formed to surround this enlarged region, as desired.

Several embodiments of tissue contourer bladders having different wall thicknesses to contour tissue in specific regions are illustrated in FIGS. 50-60.

FIGS. 50 and 51 illustrate a tissue contourer bladder 5000 having the same overall shape as the contourer of FIGS. 34-37 above. It has a bottom wall 5002 and top wall 5004 that differ in thickness. The top wall 5004 is thinner than the bottom wall, causing bladder 5000 to expand and distend to a greater degree on its upper surface than on its lower surface.

When bladder 5000 is filled with fluid, its bottom wall 5002 will remain relatively flat and will not increase substantially in size, while its thinner top surface 5004 will expand and increase in size.

FIGS. 52 and 53 illustrate another tissue contourer bladder 5200 in which expansion in controlled by varying the wall thickness. The tissue contourer bladder shown in cross-section in FIGS. 52 and 53 has the same shape and configuration as that of FIGS. 27 and 28 but provides the controlled expansion of top wall 5202 (i.e. wall 2708 in FIGS. 27 and 28) by making top wall 5202 thinner than the sidewalls 5204 and 5206, and bottom wall 5208.

FIGS. 54-56 illustrate another embodiment of the tissue contourer bladder 5400 in which the expansion and distension of the bladder is concentrated in its central region by making the bladder thinner-walled along its length than at its ends. In this example, bladder 5400 has the same overall shape as the bladder of FIG. 29: that of an extended right circular cylinder. The ends 5402 and 5404 of bladder 5400 are flat and at right angles to the cylindrical length of bladder 5400. They are thicker than the cylindrical portion 5406 of bladder 5400 and therefore resist expansion and outward distention when the bladder 5400 is filled.

The effect of this increased thickness at the ends of the bladder 5400 is illustrated in FIG. 56, which shows the shape of bladder 5400 when it is filled with fluid. Bladder 5400 expands, but the expansion occurs predominately in its central cylindrical portion 5406 and much less at each end 5402 and 5404.

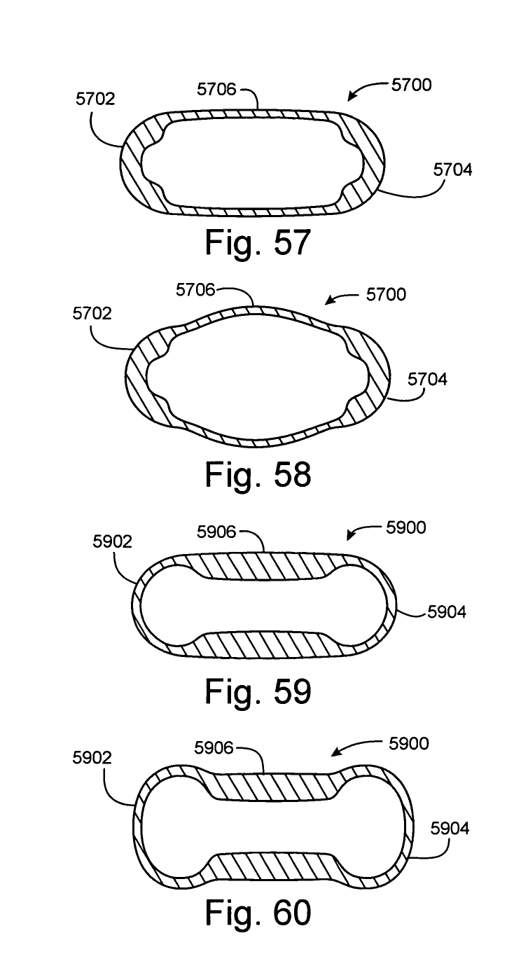

FIGS. 57 and 58 illustrate an alternative tissue contourer bladder 5700 having the same shape as that of the bladder of FIGS. 38-41, but with alternative wall thicknesses that control its expansion in a different manner.

In the example of FIGS. 38-41, the bladder has a relatively constant wall thickness, causing the bladder to expand with relatively constant cylindrical diameter along its length and to have hemispherical ends with generally the same radius.

In FIG. 57, bladder 5700 is shown in an unexpanded configuration before fluid is introduced. The ends of the bladder are thicker to provide more stiffness and rigidity to bladder ends 5702 and 5704, thereby constraining and limiting their expansion. The cylindrical sidewall 5706 of bladder 5700 has thinner walls to provide enhanced expansion of the cylindrical sidewall 5706.

FIG. 58 illustrates the effect of this configuration. In FIG. 58, the bladder 5700 of FIG. 57 has been filled with fluid and the central cylindrical sidewall 5706 has expanded significantly more than the ends 5702 and 5704.

FIGS. 59 and 60 illustrate a bladder 5900 having the inverse expansion relationship between the central region and the ends as compared to that shown in FIGS. 57 and 58. In FIGS. 59 and 60, the desired tissue expansion is at the ends of the bladder 5900. For this reason the wall thicknesses of the ends 5902, 5904 of bladder 5900 in FIGS. 59 and 60 is reduced and the wall thickness of the central cylindrical portion 5906 of bladder 5900 is increased. The thickness of central cylindrical region 5906 is greater than the ends 5902 and 5904.

By making the central cylindrical region 5906 thicker than ends 5902 and 5904, expansion and distention of the bladder occurs largely at ends 5902 and 5904, as shown in FIG. 60, than in the central cylindrical region 5906 which illustrates the bladder 5900 after it has been filled with fluid and expanded.

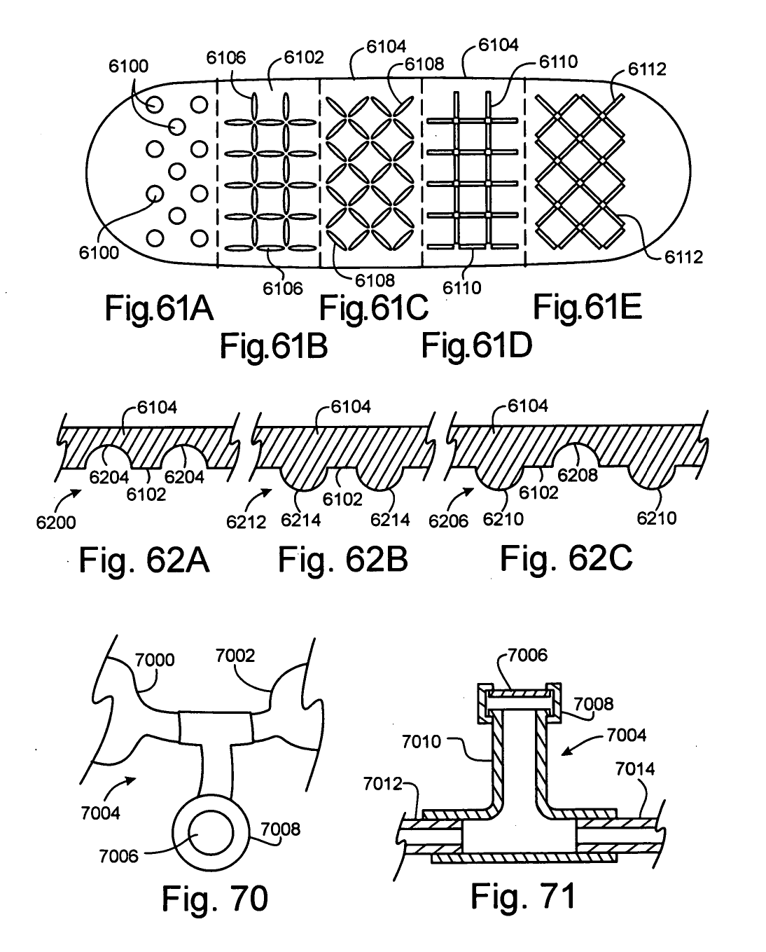

FIGS. 61A, 61B, 61C, 61D, and 61E show the different surface textures that may be applied to any of the tissue contourer bladders described or illustrated herein. Each of FIGS. 61A-61E show a different texture that can be provided on the bladders to provide a better grip on the tissue and bone inside the submucosal or subperiostial void in which they are inserted. While each of the different textures is shown as extending only over a narrow band along the length of the tissue contourer bladder, this arrangement is for ease of illustration. In the preferred embodiment, any of the textures will extend substantially the entire length of a bladder as the textures do as a whole in FIGS. 61A-61E.

The first texture illustrated is shown in FIG. 61A, which has a texture comprised of a pattern of surface irregularities 6100 shown here as generally circular protrusions or concavities disposed on a surface 6102 of a tissue contourer bladder 6104. The irregularities 6100 are preferably arranged on the surface to form polygons, preferably regular polygons, more preferably quadrilaterals, and more preferably squares, as shown here. While the pattern is shown here covering one fifth of the surface at the leftmost end, it may cover as much as the entire surface, excluding the other textures. It is preferably disposed on one side of the bladder, as shown here. In an alternative embodiment, it may extend around the circumference of the bladder.

The second texture illustrated is shown in FIG. 61B, which has a texture comprised of a pattern of surface irregularities 6106 shown here as generally oval, rounded elongate, or lens-shaped protrusions or concavities disposed on a surface 6102 of a tissue contourer bladder 6104. The irregularities 6106 are preferably arranged on the surface to form polygons, preferably regular polygons, more preferably quadrilaterals, and more preferably squares, as shown here. While the pattern is shown here covering about one-fifth of the surface adjacent the left end of the bladder, it may cover more of the surface, even the entire surface. It is preferably disposed on one side, as shown here. In an alternative embodiment, it may extend around the circumference of the bladder.

The irregularities 6106 are preferably laid end-to-end, such that the termination point of one irregularity is the beginning of another. The irregularities are preferably overlaid to define what appears to be a webwork or weave of irregularities on the surface 6102.

FIG. 61D differs from FIG. 61B in that the irregularities 6110 of FIG. 61D are elongated polygons and not elongated ovals as irregularities 6106 are. In all other respects, the patterns of FIGS. 61B and 61D are the same.

Another alternative texture is shown in FIG. 61C, in which at last some of the irregularities 6108 are arranged at right angles to each other, with a first plurality arranged diagonally to the length of the bladder and a second plurality also arranged diagonally to the length of the bladder but wherein the first and second pluralities are arranged at right angles to one another. Other than this diagonal arrangement, the irregularities and textures of FIGS. 61B and FIG. 61C are the same.

FIG. 61E differs from FIG. 61C in that the irregularities 6112 of FIG. 61E are elongated polygons and not elongated ovals as irregularities 6108 are. In all other respects, the patterns of FIGS. 61C and 61E are the same.

FIGS. 62A-62C illustrate three possible arrangements of the irregularities of FIGS. 61A-61E in the wall of bladder 6104. FIGS. 62A-C are three different alternative cross-sections of the surfaces of the bladders shown in FIGS. 61A-61E, wherein the cross-sections taken generally through the irregularities themselves.

In FIG. 62A, the irregularities 6200, which show the cross-sectional profile of the irregularities 6100, 6106, 6108, 6110, and 6112 shown in FIGS. 61A-E, all extend inwardly, forming concavities 6204 in the surface 6102 of bladder 6104 (also shown in FIGS. 61A-61E). Irregularities 6200 preferably have curved and generally hemispherical bottom.

In FIG. 62C, which illustrates another alternative arrangement of the irregularities of FIGS. 61A-61E, irregularities 6206, which show an alternative cross-sectional profile of the irregularities 6100, 6106, 6108, 6110, and 6112 shown in FIGS. 61A-E, include a combination of inwardly extending concavities 6208 and outwardly extending protrusions 6210 that are interspersed with one another in the surface 6102 of bladder 6104. Irregularities 6208 and 6210, both protrusions and concavities are preferably curved and generally hemispherical in cross-section.

In FIG. 62B, which illustrates a third alternative arrangement of the irregularities of FIGS. 61A-61E, irregularities 6212, which may be any of the irregularities 6100, 6106, 6108, 6110, and 6112 shown in FIGS. 61A-E, comprise outwardly extending protrusions 6214 in the surface 6102 of bladder 6104. Irregularities 6212 preferably have curved and generally hemispherical top surfaces in cross-section.

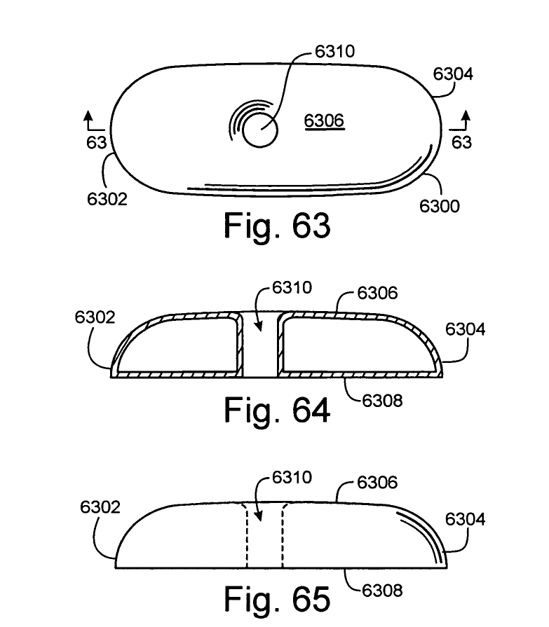

FIGS. 63-65 illustrate an alternative tissue contourer bladder having a generally vertically-extending wall that defines a passageway therethrough.

In plan view (FIG. 63) bladder 6300 has a first end 6302, a second end 6304, a top surface 6306 and a bottom surface 6308. Each end 6302 and 6304 is preferably rounded in plan view. Top surface 6306 is generally rounded at its edges and extends across and above a preferably planar bottom surface 6308. A passageway 6310 extends through the bladder 6300 from the top surface to the bottom surface. With this passageway, the bladder is in the form of a hollow torus having a single hole passing therethrough.

The passageway 6310 is generally circular and has a diameter of between 2.5 and 10.0 mm. It is sized to receive dental implant, permitting them to be inserted into the bone and extend upward through the passageway.

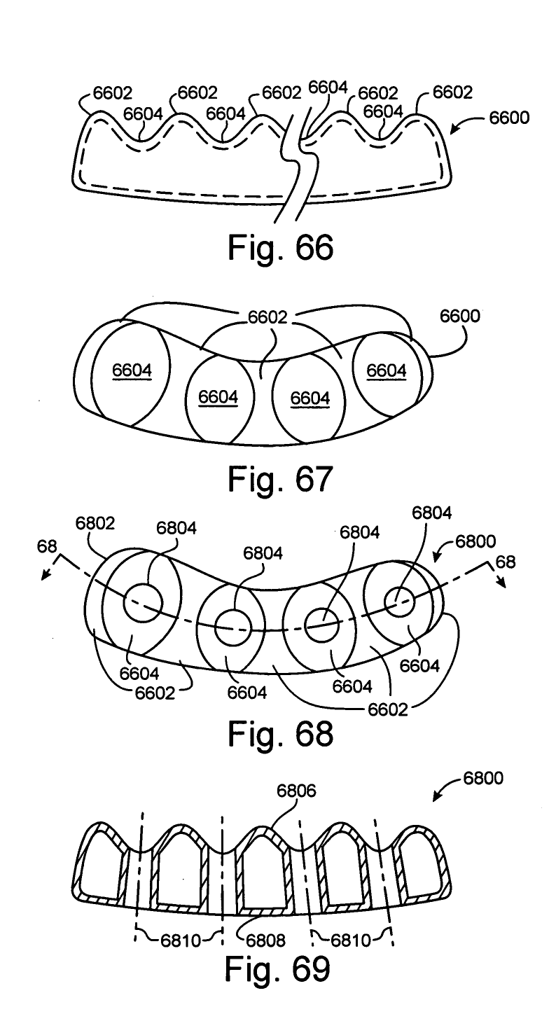

In FIGS. 66-67 a tissue contourer 6600 is shown having contours on its upper surface that imitate the contours of a healthy gum line. These contours include contouring projections 6602 that extend upward at locations corresponding to papilla. Between each of these projections are valleys or recesses 6604 that are located where missing teeth are to be replaced. The spacing of these valleys (and hence the spacing of the projections) is preferably between 2 and 10 mm. The height of the projections is preferably between 1 and 5 mm, from peak to valley.

FIGS. 68 and 69 show an alternative tissue contourer 6800 to that of FIGS. 66-67. The contourer of FIGS. 68 and 69 has a bladder 6802 with a plurality of generally vertical passageways 6804 extending therethrough. These passageways extend from the top surface 6806 to the bottom surface 6808 and are configured to receive dental implants. In all other respects, the contourer is that of FIGS. 66 and 67. Passageways 6804 have generally parallel longitudinal axes and are arranged in a line that curves to follow the crest of the mandible and maxilla. The longitudinal axis of each passageway is preferably disposed one tooth-space (e.g. between 2 and 10 mm) from the longitudinal axis of the adjacent passageways. The longitudinal axes of the passageways on each end of the contourer are preferably spaced one-half of a tooth-spacing from the end.

FIGS. 70 and 71 illustrate an alternative port arrangement for any of the tissue contourer bladders described or illustrated herein in which two bladders 7000, 7002 are coupled to a common port 7004 to be jointly and simultaneously filled and emptied. Bladders 7000 and 7002 may be any of the bladders described or illustrated herein. Port 7004 includes a self-sealing membrane 7006 that extends across the opening of annulus 7008. Annulus 7008, in turn is in fluid communication with tube 7010, which has two branches 7012 and 7014 that are in fluid communication with bladders 7000 and 7002.

The operator can insert fluid through membrane 7006 using one of the syringes or needles described herein. This fluid will traverse both branches 7012 and 7014 and respectively fill bladders 7000 and 7002, respectively.

Dimensions of the Contourer

Successfully contouring mucosal tissue depends upon several factors, including (1) the number of teeth in the edentulous gap, (2) the particular type and contour of the teeth missing in the edentulous gap, (3) the particular devices used to replace the missing teeth and bone, for example, a bone graft, mesh scaffolding or other subperiostial or submucosal structures, (4) the degree of mandibular or maxillary bone erosion, (5) the amount of suturing, and (6) the type of sutures that are used.

Experience and analysis has revealed that determining the proper bladder is not an easy task, in view of the non-linearity of certain other phenomena as well, such as the width of the edentulous gap. For example, as the width of the edentulous gap increases, the degree of bone erosion is accentuated in the medial portions of the gap. The medial portions, lacking the influence of adjacent teeth, erode at a much greater rate that is much more rapid than the erosions at the ends of the edentulous gap. Further complicating the configuration of the tissue expander are differences between anterior erosion and posterior erosion. Erosion is more pronounced in the anterior portion of the mandible and maxilla than it is in the posterior portions, either left lateral or right lateral. Thus, a tissue expander appropriate for crafting replacements to missing anterior teeth is preferably configured differently than a tissue expander appropriate for crafting replacements to missing posterior teeth.

There are three primary differences between tissue contourers, including differences in volume, differences in thicknesses, and differences in length. Of these three, two are perhaps the most significant: differences in volume and differences in length. “Length” in this context means installed length—i.e. the length of the tissue contourer when it is installed in an edentulous gap.

These differences are illustrated in the following tables, which distinguish tissue contourers by their length and their volume.

Table 1 lists the preferred range of lengths and volumes of tissue contourers used in cases of moderate maxillary and mandibular atrophy.

Table 2 lists the preferred range of lengths and volumes of tissue contourers used in cases of severe maxillary and mandibular atrophy.

Table 3 lists the preferred range of lengths and volumes of tissue contourers used in cases of extreme maxillary and mandibular atrophy.

The tables use a particular notation to indicate the specific edentulous gap they are intended to fill. The lengths of the contourers vary with the specific gap they are intended to fill. The volumes vary as well. Furthermore, the volume of each tissue expander varies with the type and degree of osseous atrophy. Table 1 identifies the characteristics of tissue contourers appropriate for contouring gaps of from one to eight teeth at different locations, and with moderate osseous atrophy. Table 2 identifies the characteristics of tissue contourers appropriate for contouring gaps of from one to eight teeth at different locations, and with severe osseous atrophy. Table 3 identifies the characteristics of tissue contourers appropriate for contouring gaps of from one to eight teeth at different locations, and with extreme osseous atrophy

Each row in the tables lists an edentulous gap, followed by a range of acceptable volumes and a range of acceptable lengths for the tissue contourer configured for that gap. The volume ranges are expressed in milliliters as maximum volumes (Vmax) and corresponding minimum volumes (Vmin) that define that preferred range. The length ranges are likewise expressed in millimeters as maximum and minimum lengths (Lmax, Lmin) that define that preferred range.

The edentulous gaps the tissue contourer is intended to contour are expressed in terms of the teeth that are missing in each gap. The teeth are identified using the Universal Dental Numbering System, in which each tooth, both maxilla and mandible, has a number of between 1 to 32, depending upon its location in the mouth of the patient.

The first row of Table 1 represents the preferred volume and length of a tissue contourer intended to contour tissue in an edentulous gap comprising a missing tooth 5, a missing tooth 12, a missing tooth 21, or a missing tooth 28. Teeth 5 and 12 are first maxillary premolars. Teeth 21 and 28 are first mandibular premolars. The second row of Table 1 represents the preferred volume and length of a tissue contourer intended to contour tissue in any of four edentulous gaps, the gaps comprising missing teeth 4 and 5, or missing teeth 12 and 13, or missing teeth 20 and 21, or missing teeth 28 and 29. These edentulous gaps correspond to both maxillary and mandibular first and second premolars.

TABLE 1
(Tissue Contourers used in Cases of Moderate Atrophy)

| edentulous gap | Vmin | Vmax | Lmin | Lmax |
|---|---|---|---|---|
| Posterior Region: Mandible & Maxilla | | | | |
| 5, 12, 21, 28 | 0.5 | 1.5 | 6.9 | 8.0 |
| 4-5, 12-13, 20-21, 28-29 | 1.0 | 3.0 | 13.6 | 15.8 |
| 3, 14, 19, 30 | 0.5 | 2.0 | 10.0 | 12.0 |
| 2-3, 14-15, 18-19, 30-31 | 1.0 | 4.0 | 19.0 | 22.4 |
| 3-5, 12-14, 19-21, 28-30 | 1.5 | 6.0 | 23.6 | 27.8 |
| 2-4, 13-15, 18-20, 29-31 | 1.0 | 4.0 | 25.9 | 30.4 |
| 2-5, 12-15, 18-21, 28-31 | 3.0 | 8.0 | 32.9 | 38.2 |
| Anterior Region: Mandible | | | | |
| 22, 27 | 0.5 | 1.5 | 6.7 | 8.0 |
| 23, 24, 25, 26 | 0.5 | 1.0 | 5.0 | 6.5 |
| 22-23, 26-27 | 1.0 | 3.0 | 13.7 | 14.5 |
| 22-24, 25-27 | 1.5 | 5.0 | 18.7 | 21.0 |
| 24-25 | 1.0 | 2.0 | 10.0 | 13.0 |
| 22-25, 24-27 | 2.0 | 6.0 | 23.7 | 27.5 |
| 22-26, 23-27 | 2.5 | 7.0 | 28.7 | 34.0 |
| 22-27 | 3.0 | 8.5 | 35.4 | 42.0 |
| Anterior Region: Maxilla | | | | |
| 6, 11 | 0.5 | 1.5 | 7.5 | 9.0 |
| 7, 10 | 0.5 | 1.0 | 6.4 | 7.0 |
| 8, 9 | 0.5 | 1.5 | 8.5 | 10.0 |
| 6-7, 10-11 | 1.0 | 3.0 | 13.9 | 16.0 |
| 6-8, 9-11 | 1.5 | 5.0 | 22.4 | 26.0 |
| 7-8, 9-10 | 1.0 | 2.5 | 14.9 | 17.0 |
| 6-9, 8-11 | 2.0 | 6.0 | 30.9 | 36.0 |
| 6-10, 7-11 | 2.5 | 7.0 | 37.3 | 43.0 |
| 6-11 | 3.0 | 8.5 | 44.8 | 52.0 |
| Transitional Region: Mandible | | | | |
| 21-24, 25-28 | 2.0 | 6.5 | 25.6 | 29.0 |
| 20-24, 25-29 | 2.5 | 8.0 | 32.3 | 36.8 |
| 19-24, 25-30 | 3.0 | 10.0 | 42.3 | 48.8 |
| 18-24, 25-31 | 4.0 | 12.0 | 51.0 | 71.2 |
| 18-22, 27-31 | 3.5 | 9.5 | 41.0 | 58.2 |
| 18-23, 26-31 | 3.5 | 10.5 | 46.0 | 64.7 |
| 21-22, 27-28 | 1.0 | 3.0 | 13.6 | 16.0 |
| 21-23, 26-28 | 1.5 | 4.0 | 18.6 | 22.5 |
| 20-22, 27-29 | 1.5 | 4.5 | 20.3 | 23.8 |
| 20-23, 26-29 | 2.0 | 5.5 | 25.3 | 30.3 |
| 19-22, 27-30 | 2.0 | 6.5 | 30.3 | 35.8 |
| Transitional Region: Maxilla | | | | |
| 5-8, 9-12 | 2.0 | 6.5 | 29.3 | 34.0 |
| 4-8, 9-13 | 2.5 | 8.0 | 36.0 | 41.8 |
| 3-8, 9-14 | 3.0 | 10.0 | 46.0 | 53.8 |
| 2-8, 9-15 | 4.0 | 12.0 | 55.0 | 64.2 |
| 2-6, 11-15 | 3.5 | 9.5 | 40.1 | 47.2 |
| 2-7, 10-15 | 3.5 | 10.5 | 46.5 | 54.2 |
| 5-6, 11-12 | 1.0 | 3.0 | 14.4 | 17.0 |
| 5-7, 10-12 | 1.5 | 4.0 | 20.8 | 24.0 |
| 4-7, 10-13 | 2.0 | 5.5 | 27.5 | 31.8 |
| 3-7, 10-14 | 2.5 | 7.5 | 37.5 | 43.8 |

TABLE 2
(Tissue Contourers used in Cases of Severe Atrophy)

| edentulous gap | Vmin | Vmax | Lmin | Lmax |
|---|---|---|---|---|
| Posterior Region: Mandible & Maxilla | | | | |
| 5, 12, 21, 28 | 0.75 | 2.25 | 6.90 | 8.00 |
| 4-5, 12-13, 20-21, 28-29 | 1.50 | 4.50 | 13.60 | 15.80 |
| 3, 14, 19, 30 | 0.75 | 3.00 | 10.00 | 12.00 |
| 2-3, 14-15, 18-19, 30-31 | 2.25 | 9.00 | 19.00 | 22.40 |
| 3-5, 12-14, 19-21, 28-30 | 2.25 | 9.00 | 23.60 | 27.80 |
| 2-4, 13-15, 18-20, 29-31 | 1.50 | 6.00 | 25.90 | 30.40 |
| 2-5, 12-15, 18-21, 28-31 | 4.50 | 12.00 | 32.90 | 38.20 |
| Anterior Region: Mandible | | | | |
| 22, 27 | 0.75 | 3.00 | 6.70 | 8.00 |
| 23, 24, 25, 26 | 0.75 | 1.50 | 5.00 | 6.50 |
| 22-23, 26-27 | 1.50 | 4.50 | 13.70 | 14.50 |
| 22-24, 25-27 | 2.25 | 7.50 | 18.70 | 21.00 |
| 24,-25 | 1.50 | 3.00 | 10.00 | 13.00 |
| 22-25, 24-27 | 3.00 | 9.00 | 23.70 | 27.50 |
| 22-26, 23-27 | 3.75 | 10.50 | 28.70 | 34.00 |
| 22-27 | 4.50 | 12.75 | 35.40 | 42.00 |
| Anterior Region: Maxilla | | | | |
| 6, 11 | 0.75 | 2.25 | 7.50 | 9.00 |
| 7, 10 | 0.75 | 1.50 | 6.40 | 7.00 |
| 8, 9 | 0.75 | 2.25 | 8.50 | 10.00 |
| 6-7, 10-11 | 1.50 | 4.50 | 13.90 | 16.00 |
| 6-8, 9-11 | 2.25 | 7.50 | 22.40 | 26.00 |
| 7-8, 9-10 | 1.50 | 3.75 | 14.90 | 17.00 |
| 6-9, 8-11 | 3.00 | 9.00 | 30.90 | 36.00 |
| 6-10, 7-11 | 3.75 | 10.50 | 37.30 | 43.00 |
| 6-11 | 4.50 | 12.75 | 44.80 | 52.00 |
| Transitional Region: Mandible | | | | |
| 21-24, 25-28 | 3.00 | 9.75 | 25.60 | 29.00 |
| 20-24, 25-29 | 3.75 | 12.00 | 32.30 | 36.80 |
| 19-24, 25-30 | 4.50 | 15.00 | 42.30 | 48.80 |
| 18-24, 25-31 | 6.00 | 18.00 | 51.00 | 71.20 |
| 18-22, 27-31 | 5.25 | 14.25 | 41.00 | 58.20 |
| 18-23, 26-31 | 5.25 | 15.75 | 46.00 | 64.70 |
| 21-22, 27-28 | 1.50 | 4.50 | 13.60 | 16.00 |
| 21-23, 26-28 | 2.25 | 6.00 | 18.60 | 22.50 |
| 20-22, 27-29 | 2.25 | 6.75 | 20.30 | 23.80 |
| 20-23, 26-29 | 3.00 | 8.25 | 25.30 | 30.30 |
| 19-22, 27-30 | 3.00 | 9.75 | 30.30 | 35.80 |
| Transitional Region: Maxilla | | | | |
| 5-8, 9-12 | 3.00 | 9.75 | 29.30 | 34.00 |
| 4-8, 9-13 | 3.75 | 12.00 | 36.00 | 41.80 |
| 3-8, 9-14 | 4.50 | 15.00 | 46.00 | 53.80 |
| 2-8, 9-15 | 6.00 | 18.00 | 55.00 | 64.20 |
| 2-6, 11-15 | 5.25 | 14.25 | 40.10 | 47.20 |
| 2-7, 10-15 | 5.25 | 15.75 | 46.50 | 54.20 |
| 5-6, 11-12 | 1.50 | 4.50 | 14.40 | 17.00 |
| 5-7, 10-12 | 2.25 | 6.00 | 20.80 | 24.00 |
| 4-7, 10-13 | 3.00 | 8.25 | 27.50 | 31.80 |
| 3-7, 10-14 | 3.75 | 11.25 | 37.50 | 43.80 |

TABLE 3
(Tissue Contourers used in Cases of Moderate Atrophy)

| edentulous gap | Vmin | Vmax | Lmin | Lmax |
|---|---|---|---|---|
| Posterior Region: Mandible & Maxilla | | | | |
| 5, 12, 21, 28 | 1.30 | 3.90 | 6.9 | 8.0 |
| 4-5, 12-13, 20-21, 28-29 | 2.60 | 7.80 | 13.6 | 15.8 |
| 3, 14, 19, 30 | 1.30 | 5.20 | 10.0 | 12.0 |
| 2-3, 14-15, 18-19, 30-31 | 2.60 | 10.40 | 19.0 | 22.4 |
| 3-5, 12-14, 19-21, 28-30 | 3.75 | 6.00 | 23.6 | 27.8 |
| 2-4, 13-15, 18-20, 29-31 | 2.60 | 10.40 | 25.9 | 30.4 |
| 2-5, 12-15, 18-21, 28-31 | 7.80 | 20.80 | 32.9 | 38.2 |
| Anterior Region: Mandible | | | | |
| 22, 27 | 1.3 | 3.9 | 6.7 | 8.0 |
| 23, 24, 25, 26 | 1.3 | 2.6 | 5.0 | 6.5 |
| 22-23, 26-27 | 2.6 | 7.8 | 13.7 | 14.5 |
| 22-24, 25-27 | 3.9 | 13.0 | 18.7 | 21.0 |
| 24,-25 | 2.6 | 5.2 | 10.0 | 13.0 |
| 22-25, 24-27 | 5.2 | 6.0 | 23.7 | 27.5 |
| 22-26, 23-27 | 2.5 | 18.2 | 28.7 | 34.0 |
| 22-27 | 7.8 | 22.1 | 35.4 | 42.0 |
| Anterior Region: Maxilla | | | | |
| 6, 11 | 1.3 | 3.9 | 7.5 | 9.0 |
| 7, 10 | 1.3 | 2.6 | 6.4 | 7.0 |
| 8, 9 | 1.3 | 3.9 | 8.5 | 10.0 |
| 6-7, 10-11 | 2.6 | 7.8 | 13.9 | 16.0 |
| 6-8, 9-11 | 3.9 | 13.0 | 22.4 | 26.0 |
| 7-8, 9-10 | 2.6 | 6.5 | 14.9 | 17.0 |
| 6-9, 8-11 | 5.2 | 15.6 | 30.9 | 36.0 |
| 6-10, 7-11 | 6.5 | 18.2 | 37.3 | 43.0 |
| 6-11 | 7.8 | 22.1 | 44.8 | 52.0 |
| Transitional Region: Mandible | | | | |
| 21-24, 25-28 | 5.2 | 16.9 | 25.6 | 29.0 |
| 20-24, 25-29 | 6.5 | 20.8 | 32.3 | 36.8 |
| 19-24, 25-30 | 7.8 | 26.0 | 42.3 | 48.8 |
| 18-24, 25-31 | 10.4 | 31.2 | 51.0 | 71.2 |
| 18-22, 27-31 | 9.1 | 24.7 | 41.0 | 58.2 |
| 18-23, 26-31 | 9.1 | 27.3 | 46.0 | 64.7 |
| 21-22, 27-28 | 2.6 | 7.8 | 13.6 | 16.0 |
| 21-23, 26-28 | 3.9 | 10.4 | 18.6 | 22.5 |
| 20-22, 27-29 | 3.9 | 11.7 | 20.3 | 23.8 |
| 20-23, 26-29 | 5.2 | 14.3 | 25.3 | 30.3 |
| 19-22, 27-30 | 5.2 | 16.9 | 30.3 | 35.8 |
| Transitional Region: Maxilla | | | | |
| 5-8, 9-12 | 5.2 | 16.9 | 29.3 | 34.0 |
| 4-8, 9-13 | 6.5 | 20.8 | 36.0 | 41.8 |
| 3-8, 9-14 | 7.8 | 26.0 | 46.0 | 53.8 |
| 2-8, 9-15 | 10.4 | 31.2 | 55.0 | 64.2 |
| 2-6, 11-15 | 9.1 | 24.7 | 40.1 | 47.2 |
| 2-7, 10-15 | 9.1 | 27.3 | 46.5 | 54.2 |
| 5-6, 11-12 | 2.6 | 7.8 | 14.4 | 17.0 |
| 5-7, 10-12 | 3.9 | 10.4 | 20.8 | 24.0 |
| 4-7, 10-13 | 5.2 | 14.3 | 27.5 | 31.8 |
| 3-7, 10-14 | 6.5 | 19.5 | 37.5 | 43.8 |

Tissue contourers may be filled with any of several fluids. These fluids may be compressible or incompressible. Acceptable compressible fluids include air, carbon dioxide, or nitrogen. Acceptable incompressible fluids include sterile water, saline solution or other biocompatible fluids. The tissue contourers may also be filled with a mixture of both a compressible fluid and an incompressible fluid.

Such a combination of fluids have been determined (1) to support the tissue that is being expanded, preventing it from being crushed or overly compressed, such as during vigorous chewing, and (2) to provide internal tension to strain the tissue even after it expands slightly. If the contourer is partially filled with compressible fluid at a pressure above atmospheric pressure, it will be able to apply a residual contouring force to the tissue being contoured.

From the foregoing, it will be observed that numerous modifications and variations can be effected without departing from the true spirit and scope of the novel concept of the present invention. For example, any of the various ports described herein may be used with any of the tissue contourer bladders described herein. Any of the tissue contourers show herein may be provided with or without ports. It will be appreciated that the present disclosure is intended as an exemplification of the invention, and is not intended to limit the invention to the specific embodiment illustrated. The disclosure is intended to cover by the appended claims all such modifications as fall within the scope of the claims.

1. A tissue contourer for contouring mucosal tissue in preparation for dental implantation or grafting, the contourer comprising:
a central portion having a first side configured to abut an edentulous portion of a maxilla or mandible and a second side configured to contour mucosal tissue overlying the edentulous portion;
a first end extending from the central portion and configured to abut a first tooth at one end of the edentulous gap;
a second end extending from the central portion and configured to abut another tooth at another end of the edentulous gap; and
a means for filling the central portion with fluid.

2. The tissue contourer of claim 1, wherein the first end, the second end and the central portion have an overall length of no more than 71.2 mm.

3. The tissue contourer of claim 2, wherein the first end, the second end and the central portion have an overall length of no more than 52 mm.

4. The tissue contourer of claim 3, wherein the first end, the second end and the central portion have an overall length of no more than 26 mm.

5. The tissue contourer of claim 2, wherein the contourer has an internal working volume of no more than 31.2 ml.

6. The tissue contourer of claim 5, wherein the contourer has an internal working volume of no more than 22.1 ml.

7. The tissue contourer of claim 6, wherein the contourer has an internal working volume of no more than 5 ml.

8. A tissue contourer bladder for contouring mucosal tissue in preparation for dental implantation or grafting, the bladder comprising:
an elongate central portion configured to be disposed between a maxilla or mandible and mucosal tissue overlying the maxilla or mandible;
a first end configured to be disposed in a first terminal gap; and
a second end configured to be disposed in a second terminal gap.

9. The tissue contourer bladder of claim 8, wherein the first and second terminal gaps are the same terminal gap.

10. The tissue contourer bladder of claim 8, wherein the first and second terminal gaps are in the same quadrant.

11. The tissue contourer bladder of claim 8, wherein the first and second terminal gaps are in adjacent quadrants.

12. A tissue contourer for contouring mucosal tissue in preparation for dental implantation or grafting, the contourer comprising a bladder having:
an elongated central portion with a texture, wherein the central portion is configured to be disposed between a maxilla or mandible and overlying mucosal tissue;
a first end configured to be disposed in a first terminal gap; and
a second end configured to be disposed in a second terminal gap.

13. The tissue contourer of claim 12, wherein the texture is defined by a plurality of protrusions extending outward away from a surface of the central portion.

14. The tissue contourer of claim 13, wherein the texture is further defined by a plurality of concavities extending inwardly from the surface of the central portion.

15. The tissue contourer of claim 12, wherein the texture is defined by a plurality of concavities extending inwardly from a surface of the central portion.

16. The tissue contourer of claim 12, wherein the texture is defined by a plurality of elongated concavities or protrusions.

17. The tissue contourer of claim 16, wherein the elongated concavities or protrusions are formed as elongated ovals.

18. The tissue contourer of claim 17, wherein each of the elongated ovals is disposed at an angle to its adjacent elongated oval.

19. A tissue contourer for contouring mucosal tissue in preparation for dental implantation or grafting, the contourer comprising:
an elongated central portion having a top side, a bottom side, a facial-facing side, and a lingual-facing side, wherein at least one of the four sides is stiffer than the other three sides;
a first end configured to be disposed in a first terminal gap at one end of an edentulous gap; and
a second end configured to be disposed in a second terminal gap at another end of the edentulous gap.

20. The tissue contourer of claim 19, wherein the bottom side is stiffer than the top side.

21. The tissue contourer of claim 20, wherein the facial-facing and lingual-facing sides are stiffer than the top side.

22. The tissue contourer of claim 19, wherein the elongated central portion is a polygon in at least one cross-section.

23. The tissue contourer of claim 20, wherein the elongated central portion is a quadrilateral in at least one cross-section.

24. The tissue contourer of claim 23, wherein the elongated central portion is a regular polygon in at least one cross-section.

25. The tissue contourer of claim 19 wherein the two ends are stiffer than the top side.

26. A tissue contourer for contouring mucosal tissue in preparation for dental implantation or grafting, the contourer comprising:
a cylindrical elongated central portion with at least one longitudinal cross-section that is a conic section;
a first end configured to be disposed in a first terminal gap at one end of an edentulous gap; and
a second end configured to be disposed in a second terminal gap at another end of the edentulous gap.

27. The tissue contourer of claim 26 wherein the conic section is a circle.

28. The tissue contourer of claim 27, wherein the conic section does not change when the tissue contourer is filled with fluid.

29. The tissue contourer of claim 26, wherein the elongated central portion is made of a self-sealing material.

30. A tissue contourer for contouring mucosal tissue in an edentulous gap in preparation for dental implantation or grafting, the contourer comprising:
a central portion configured to contour mucosal tissue overlying the edentulous gap;
a first end configured to be disposed in a first terminal gap at one end of the edentulous gap; and
a second end configured to be disposed in a second terminal gap at another end of the edentulous gap; and
a means for filling the central portion with fluid.

31. The tissue contourer of claim 30, wherein the means for filling the central portion with fluid includes a membrane means for limiting fluid leakage from the contourer.

32. The tissue contourer of claim 31, wherein the means for filling the central portion with fluid includes an annulus defining an opening through which fluid is introduced into the central portion.

33. The tissue contourer of claim 32, wherein the means for filling the central portion includes a means coupled to and between the annulus and the central portion to direct fluid from the annulus to the central portion.

34. A tissue contourer for contouring mucosal tissue in an edentulous gap in preparation for dental implantation or grafting, the contourer comprising:
a central portion configured to contour mucosal tissue overlying the edentulous gap;
a first end coupled to the central portion and configured to be disposed in a first terminal gap at one end of the edentulous gap; and
a second end coupled to the central portion configured to be disposed in a second terminal gap at another end of the edentulous gap.

35. The tissue contourer of claim 34, wherein the central portion has a top surface and wherein the central portion further comprises a plurality of projections disposed on the top surface and extending upwardly therefrom.

36. The tissue contourer of claim 35, wherein the plurality of projections is spaced between 2 and 10 mm apart.

37. The tissue contourer of claim 36, wherein the plurality of projections are disposed to contour papillae in the mucosal tissue.

38. The tissue contourer of claim 36, wherein the plurality of projections extend between 1 and 5 mm upward from the top surface.

39. A tissue contourer for contouring mucosal tissue in an edentulous gap in preparation for dental implantation or grafting, the contourer comprising:
a bladder having a central portion configured to contour mucosal tissue overlying the edentulous gap, a first end configured to be disposed in a first terminal gap at one end of the edentulous gap, and a second end configured to be disposed in a second terminal gap at another end of the edentulous gap,
wherein the bladder defines at least one passageway extending therethrough.

40. The tissue contourer of claim 40 wherein the passageway extends from a top surface through a bottom surface of the bladder.

41. The tissue contourer of claim 1, wherein the means for filling is selected from the group consisting of a self-sealing portion of central portion and a port.
